(19) United States
(12) Patent Application Publication
(10) Pub. No.: US 2021/0039070 A1
(43) Pub. Date: Feb. 11, 2021

(54) COMPOSITE MATERIALS FOR THE DEPLETION OF CONTAMINANTS FROM SOLUTIONS

(71) Applicant: Klawego GmbH & Co. KG, Heddesheim (DE)

(72) Inventors: Klaus Gottschall, Heddesheim (DE); Lothar Britsch, Reute (DE); Evelyn Gottschall, Heddesheim (DE)

(21) Appl. No.: 16/977,237

(22) PCT Filed: Mar. 15, 2019
(86) PCT No.: PCT/EP2019/056600

§ 371 Date: Sep. 1, 2020

(30) Foreign Application Priority Data
Mar. 15, 2018 (EP) 18161976.8

(51) Int. Cl. B01J 20/26; C07K 1/16; B01J 20/10; B01J 20/289; B01J 20/28; B01J 20/32; B01J 20/30

(52) U.S. Cl. CPC B01J 20/267; C07K 1/16; B01J 20/265; B01J 20/103; B01J 20/289; B01J 2220/54; B01J 20/327; B01J 20/3085; B01J 2220/445; B01J 2220/46; B01J 20/28083

(57) ABSTRACT

Composite material comprising a support material and at least one polymeric layer, wherein the at least one polymeric layer is present in form of a polymeric mesh and is comprising at least one non-adsorbing/non-adsorptive polymer with respect to a target compound, and wherein said composite material further comprises sites which are adsorbing/adsorptive for an impurity compound; and a combination of at least one first adsorbent and at least one second adsorbent, wherein the at least one first adsorbent comprises at least one composite material comprising at least one adsorbing/adsorptive polymer, or at least one non-adsorbing/non-adsorptive polymer, or at least one adsorbing/adsorptive polymer together with at least one non-adsorbing/non-adsorptive polymer.

### TECHNICAL FIELD

The invention relates to the provision of materials, processes and methods allowing the complete separation of various impurities comprising different chemical constitutions from a complex feedstock.

### BACKGROUND OF THE INVENTION

Purified soluble macromolecules are very important substances throughout the industries. Mainly the pharmaceutical and medical areas are reporting a growing need for bio-polymeric substances, primarily for therapeutic and diagnostic purposes, but also for technologies like tissue engineering.

The separation of bio-polymers from the raw process solutions is usually achieved with chromatographic methods. Due to the huge number of different impurities in raw solutions of macromolecules, e.g. crude extracts from almost all kinds of biological starting materials, particularly those from living or dead tissues, tissue and cell cultures of various cultivation techniques, the first step in a conventional chromatographic purification process is usually comprising the binding of the target compounds (“capture”), whereas the majority of undesired products is left unbound at all or may be separated from the target by a selective elution step, releasing bound impurities before or after the target substance during this step. It would be highly advantageous, however, in terms of product recovery and overall process streamlining, to bind the majority of impurities in a first step, whereas the purified target compound remains unbound in the solution.

PCT/EP2017/073332 teaches how a macromolecular target compound, preferably an antibody, is purified and how impurities are depleted from a complex feedstock in one batch separation step, using a polymeric mesh, whereas at least one purified target compound was excluded from said mesh and thus remaining in the supernatant, while the majority of impurities was retained by the adsorbent. Said polymeric mesh is preferably comprising a cross-linked polyamine, either a composite material or a soft gel.

The present application is providing a variety of materials and synthesis processes, based on a different composite design and a multitude of different functional polymers. Thus, an excellent selectivity is achieved regarding the separation of impurities and contaminants of various chemical structures. Also the recovery of the target compound is excellent. Finally, a very high phase volume ratio between the liquid phase and the solid phase is accomplished in separation processes, enabling attractive new opportunities in large scale operations.

### SUMMARY OF THE INVENTION

The technical object of the present application is to provide materials, processes and methods allowing the complete separation of various impurities comprising different chemical constitutions from a complex feedstock, preferably without binding the target compound, favourably applying a one-step process.

The present invention is relating to materials as follows: Composite materials comprising a number of 0, 1, or 2, or 3, or 4 . . . or n of adsorptive polymers and a number of n+1, or n+2, or n+i non-adsorptive/non-adsorbing polymers, whereas at least one non-adsorptive/non-adsorbing polymer is attached on the boundary surface of the ultimate binding layer or on the outer surface of the support material.

Composite materials for the separation of at least one target compound from at least two impurities, comprising at least two adsorptive polymers, each of them binding at least one distinct impurity originating from a feedstock, whereas the part of the surface of the composite, which is accessible for at least one target compound, does not bind said target compound, which is optionally prevented from access into the composite mesh by size exclusion.

A mixture of at least two adsorbents, comprising at least one composite material, whereas at least one of these composite materials is equipped with at least one adsorptive polymer.

A mixture of at least two composite adsorbents, comprising at least two different adsorptive polymers, whereas at least one of said composite adsorbents may be optionally equipped with one non-adsorptive polymer.

The present invention is relating to methods and processes as follows: Methods comprising the chemical constitution of a polymeric layer together with the composition and the pH of a liquid, thus enabling the size exclusion of pullulane standards and target compounds of a distinct hydrodynamic radius Rₕ from a composite adsorbent.

Methods for the selective derivatisation of the functional groups of a polymer comprised in a composite material.

The invention is defined in the claims appended to the description.

### GENERAL DESCRIPTION

Design and Materials

The following embodiments and descriptions are comprising the principles for materials design and the related terms.

The materials of the present invention are comprising composite materials, characterized in that at least one polymeric layer is immobilized to a support material, said layers altogether forming a polymeric mesh.

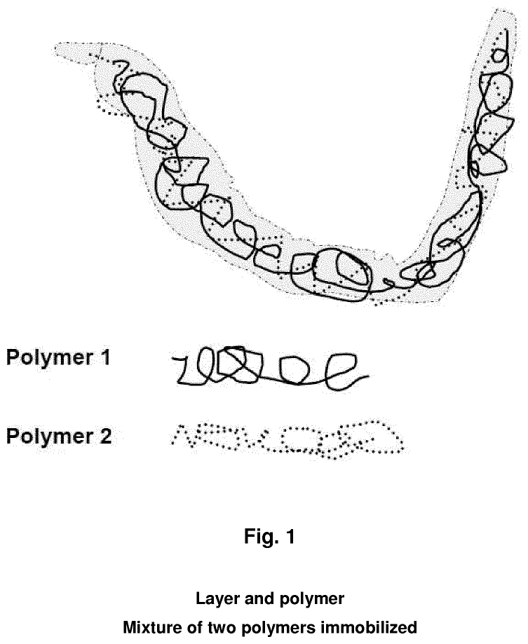

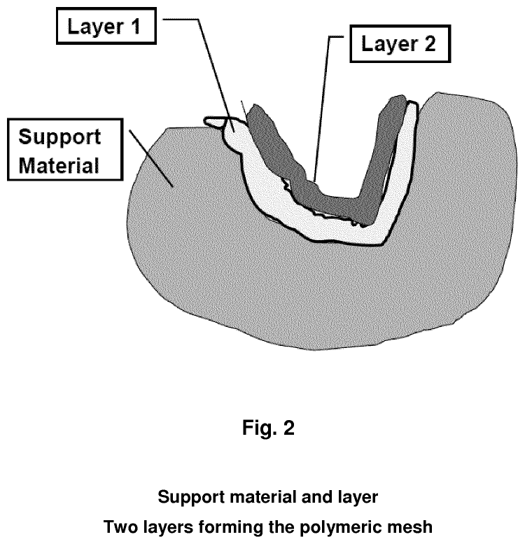

In one preferred embodiment, in combination with any of the above or below embodiments, at least one polymer is immobilized within at least one layer on at least a part of the support surface (FIG. 1) in at least one step of preparation, thus forming a mesh comprising at least one discrete layer of surface coating (FIG. 2).

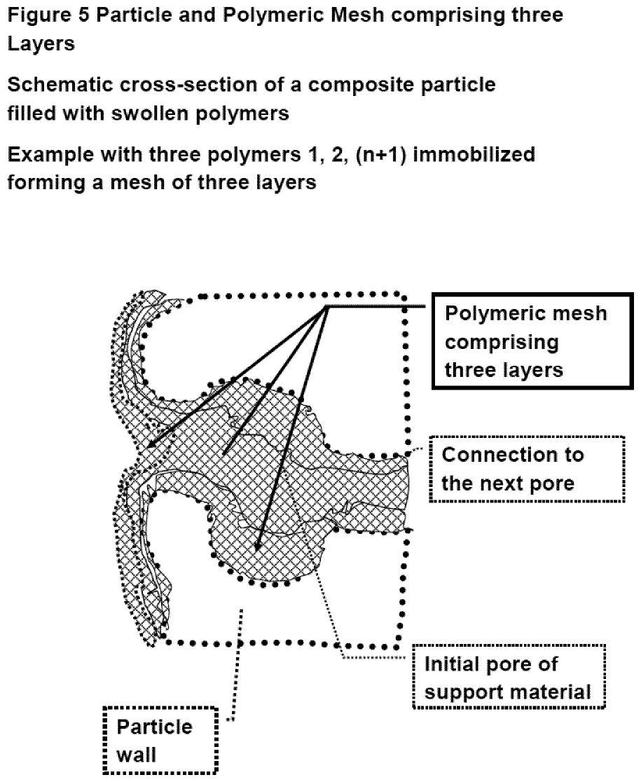

Accordingly a polymeric mesh (FIG. 5 shows one embodiment) is comprising at least one layer, which in turn is comprising at least one polymer.

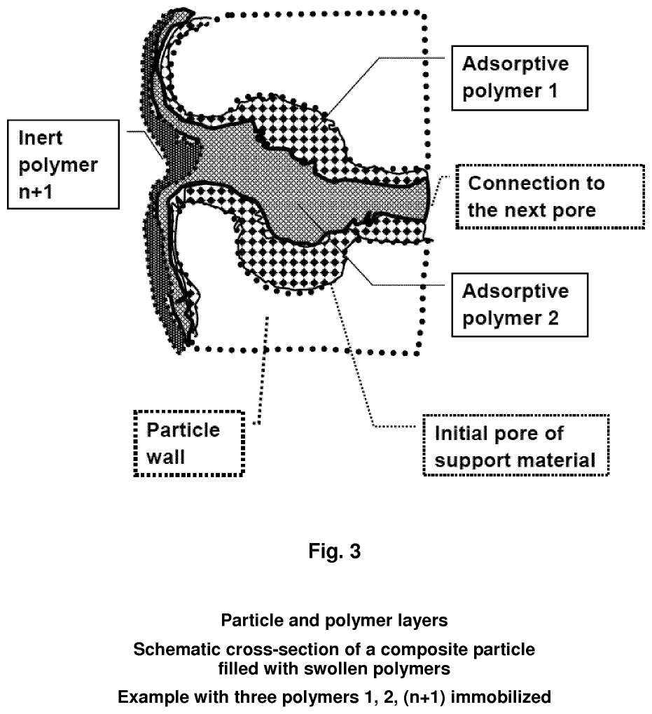

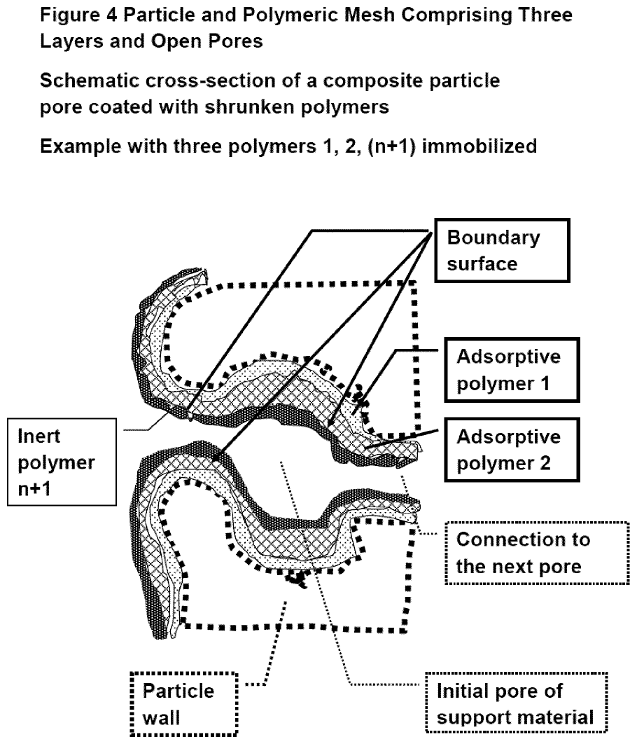

The lastly immobilized layer, as a part of said mesh, is either covering the exterior surface of a support material or composite particle (FIG. 3 shows one embodiment), or is covering the boundary surface of the earlier attached polymeric layers inside and outside a support pore (FIG. 4). This lastly immobilized layer is comprising at least one non-adsorptive polymer n+1.

Alternatively the non-adsorptive properties are introduced by the partial selective derivatisation of the outer surface of the layer attached with the previous immobilization step, as described in the chapter Derivatisation below.

In this context it is important to realize, that the composite materials of the present invention are comprising two different kinds of porosity of different origin: Firstly, there is the intrinsic porosity of the immobilized polymer globules or coils in the range of less than 10 nm, as determined by inverse Size Exclusion Chromatography (iSEC, see section Methods), calibrated using molecular size standards owing hydrodynamic radii (Rₕ) below 10 nm. This pore fraction is significant for the polymer in swollen state. Secondly there is a remaining porosity of the support material, as long as the immobilized polymer coils and globules do not completely fill in the support pore volume. As polymers usually are exhibiting a broad molecular mass distribution, hence broadly distributed molecular sizes, also moieties with a mass below 2000 Da may occur in the polymer solution used for immobilization purposes. Thus, those small sized polymer molecules may penetrate the pores of the vicinal polymer layers to a certain extent.

A layer is defined as the portion of at least one polymer which was immobilized in one step of preparation. The boundary surface between the previously attached layer and the layer attached with the subsequent step is the site where these two layers are contacting each other. They may also slightly permeate each other. A polymeric mesh (FIG. 5) is comprising at least one layer. The extension and porosity of the particular polymers or layers is determined using iSEC, utilizing appropriate solvents, as based on the known swelling behaviour of the individual polymers.

Provided that one layer is comprising an amino polymer, it is swelling in acidic solvents due to the electrostatic repulsion of protonated functional groups. In contrast, a polymer bearing carboxylic groups will remain shrunken under those conditions. In presence of basic solvents the situation appears generally reversed.

In this way the partial volume of immobilized polymers and layers is determined for each particular intermediate product of the composite synthesis. For details see the section Morphology below.

The preferred strategy to prepare composite designs according to FIG. 3 is comprising the filling of the pores with at least one polymer solution, swelling said polymer after immobilization and evaporation of the solvent, whereas the final polymer is excluded from the pores of the composite under the solvent and pH-conditions of the synthesis. For details see the embodiments in the synthesis section below. The accessibility of the pores is hereby determined by inverse Size Exclusion Chromatography (iSEC) and HPLC under standardized conditions. The final polymer, which should remain excluded is hereby used itself as a test probe. In contrast, the embodiments according to FIG. 4 are preferably realized with large pore support materials and polymers in shrunken state, whereas the polymer, which is finally immobilized, does not significantly intrude into the pores of the penultimate layer under the conditions of preparation. For this reason preferably lastly immobilized polymers with a small content below a certain molecular mass are applied, preferably without a fraction below 5,000 Da, more preferred below 20,000 Da, most preferred below 50,000 Da.

Different polymers with different structure, mainly different functional groups are the necessary elements of the present invention. They are indicated as follows: 0, 1, 2, 3, . . . n represent the number of adsorptive or binding polymers, with respect to the impurities, whereas n+1, n+2 etc. represent the number of non-binding polymers with respect to the target compound.

The terms adsorption and binding and non-covalent interaction are used as synonyms throughout the present application. Affinity is a synonym for the potential binding of a particular substance or group of chemically related substances by an adsorbent, and is correlated with the partitioning of each particular substance between the two phases solid and liquid, as expressed by the partitioning coefficient P.

Different kind of affinity means, that the composite adsorbent exhibits at least two different structures, capable of complementary interaction with at least two different classes of impurities or contaminants. Ionic ligands binding impurities of different net charge as expressed by their isoelectric point are one very common example.

Chemically related compounds, substances, sites or materials are exhibiting the same or similar structural elements or functional groups, in particular residues contributing a major portion to the binding enthalpy towards a certain epitope or the same epitope of the adsorbent or receptor.

Class of impurities means a number of compounds which are chemically related.

Adsorptive in the context of the present invention means, that in the solvent of application the partitioning coefficient P between the solid phase and the liquid phase has a certain minimum value. P is for a particular compound at least 3, preferably at least 5, more preferred at least 10, most preferred greater than 50. In this case the adsorbent has a certain affinity towards substances to be bound.

The partitioning coefficient P is defined as

P=C_{solid}/C_{liq}

C_{solid} is the equilibrium concentration of said compound in the solid phase.

c_{liq} is the equilibrium concentration of said compound in the liquid phase.

Inert or non-adsorptive or non-adsorbing means that the partitioning coefficient P between the solid phase and the liquid phase is small. The partitioning coefficient P is preferably below 0.2, more preferred below 0.05, most preferred below 0.02.

Retained by the composite adsorbent means the depletion inside of the mesh pores, due to any non-covalent or covalent binding mechanism like adsorption, or due to a partitioning, size exclusion, or extraction mechanism.

Polymer

For the purpose of the present invention any polymer is applicable, either soluble in aqueous or organic liquids, and capable of derivatisation and cross-linking reactions. A polymer suspension dissolving during these chemical steps is considered also useful for the purpose of the present invention.

In combination with any of the above or below embodiments, the average molecular weight of the polymer is preferably 2,000 to 2,000,000 Dalton, more preferably 10,000 to 1,000,000 Dalton, even more preferably 15,000 to 200,000 Dalton, most preferably 20,000 to 100,000 Dalton.

In a preferred embodiment, in combination with any of the below embodiments, the cross-linkable polymer or co-polymer molecules are comprising at least one functional group (a “functional polymer”).

Basically the functional polymer may be any kind of polymer comprising at least one or more identical or different functional groups.

Preferably the functional polymer is bearing at least one OH—, SH—, COOH—, —SO₃H, —PO₄H₂, —PO₃H, epoxy, or primary or secondary amino group.

Co-polymers, polycondensation products (e.g., polyamides), and oligomers or molecules with at least four equal or different repetitive units are considered within the polymer definition for the present invention.

In a preferred embodiment, in combination with any of the above or below embodiments, the functional polymer is an amino group containing polymer (“a polyamine”), or an oligomer with at least four amino groups. Amino groups are primary and secondary.

Among polyamines are more preferred: poly(vinylformamide-co-vinylamine), linear or branched poly(vinylamine), poly(allylamine), poly(ethyleneimine), poly-lysine, or copolymers containing such amino polymers.

Most preferred is the composition of poly(vinylformamide-co-vinylamine) comprising 5% to 80% of poly(vinylformamide), preferably 10% to 40%, more preferred 10% to 20%.

In a further preferred embodiment, in combination with any of the above or below embodiments, the polyamine is a mixture of a poly(vinylamine) and poly(vinylformamide-co-vinylamine).

Other preferred polymers are mentioned together with the related embodiment or within the explanations.

Within a preferred embodiment, in combination with any of the below embodiments, technical grade, raw functional polymers and solutions thereof are used in order to synthesize the composite adsorbent.

Preferably raw poly(vinylamine) or poly(vinylformamide-co-vinylamine) solution is used, containing the salts, sodium hydroxide, sodium formate, and other side products from the polymer manufacturing process (Example 1).

As the low molecular weight impurities and side-products of said technical grade polymers in general are easily washed out after the polymer immobilisation, the final composite material exhibits a high purity.

Support Material

Any support material can be used for the preparation of the composite materials of the present invention, provided that a first polymer immobilized to the support surface remains stabile under the conditions of preparation, rinsing, cleaning and application. Preferred support materials are particulate materials with an average particle size of 3 μm to 10 mm, preferably between 20 μm and 500 μm, most preferred between 35 μm and 200 μm.

Applicable are average pore sizes of 2 nm to 5 mm, preferred are pore sizes between 5 nm and 500 nm, more preferred is the range between 10 nm and 100 nm, most preferred between 15 nm and 30 nm determined with the usual methods as applied by the manufacturers. The form of the porous support material is not particularly limited and can be, for example, a membrane, a non-woven tissue, a monolithic or a particulate material.

Particulate and monolithic porous materials are preferred as the support. The shape of the particulate porous support material can be either irregular or spherical. In combination with any of the above or below embodiments, the porous support material preferably has a substantially irregular shape.

Monolithic means a homogeneously porous piece of support material exhibiting a thickness of at least 0.5 mm. In a further preferred embodiment, in combination with any of the above or below embodiments, the monolithic support material is a disk, a torus, a cylinder or a hollow cylinder, with at least 0.5 mm height and with an arbitrary diameter.

Pellicular materials are also within the scope of the present invention. Pellicular materials are commercially available comprising solid particles coated with a porous layer.

In a preferred embodiment, in combination with any of the above or below embodiments, the porous support materials are composed of a metal oxide, a semimetal oxide, ceramic materials, zeolites, or natural or synthetic polymeric materials.

In a further preferred embodiment, in combination with any of the above or below embodiments, the porous support material is porous cellulose, acetyl cellulose, methyl cellulose, chitosane or agarose.

Most preferred is cellulose and acetylcellulose, either particles or monolithic.

In a further preferred embodiment, in combination with any of the above or below embodiments, the porous support material is a porous polyacrylate, polymethacrylate, polyetherketone, polyalkylether, polyarylether, polyvinylalcohol, or polystyrene.

In a further preferred embodiment, in combination with any of the above or below embodiments, the support material is silica, alumina or titanium dioxide with an average pore size (diameter) between 20 nm and 100 nm (as analyzed by mercury intrusion according to DIN 66133) and a surface area of at least 100 m2/g (BET-surface area according to DIN 66132).

In a further preferred embodiment, in combination with any of the above or below embodiments, the support material is irregularly shaped silica, alumina or titanium dioxide, with a surface area at least 150 m2/g.

Even more preferred are irregularly shaped silica gel materials, exhibiting an average pore diameter of 20-30 nm, and other support materials from the above list, allowing the access of polymeric pullulane standards with a hydrodynamic radius Rₕ below or equal to 6 nm, when dissolved and measured under iSEC conditions in 20 mM ammonium acetate at pH 6.

Most preferred is irregular silica with a BET surface area of at least 150 m2/g, preferably 250 m2/g and a pore volume (mercury intrusion) of at least 1.5 ml/g, preferably 1.8 ml/g.

Designing the morphology of a polymeric mesh, comprised in a composite adsorbent.

Pore size, exclusion limit, swelling and shrinking.

The following remarks and considerations are summarizing the stereo-chemical properties of the composite adsorbent as comprised in the present invention. These issues are critical for both their preparation and their use.

If polymers are immobilized inside the pores of a support material, they do not display any observable mesh porosity in a dry state. After drying such a composite, approximately the pore size distribution of the basic support material is found again, using the established methods like BET nitrogen adsorption or mercury intrusion porosimetry, at least as long as the degree of cross-linking remains below 25%.

This behaviour may be attributed to the strong adhesive forces inside the polymer coils, causing shrinkage of the mesh to values close to the excluded polymer volume. If the functional groups are bearing a charge as it is possible, e.g., with polyacrylate or a polyamine, the resultant excluded volume in the dry stage may be slightly bigger.

If wetted by a solvent or in a suspension the polymer structure displays a fundamentally different morphology. Provided sufficient solvation, the polymer mesh swells until reaching the maximal possible volume, spanning a classical hydrogel structure.

In this case, the resultant porosity of the polymeric mesh is dependent on the nature of the solvent (polarity, etc.), the pH, ionic strength and the concentration of auxiliaries like detergents.

When treating functional polymers, in particular charged polymers, according to the present invention, it is important to distinguish between the degree of filling the support material pores whilst the process of immobilization of the polymer, and “filled or occupied pores” whilst the use of the composite in separations.

In the first case of polymer attachment, the entire support pore volume is filled with a solution of the reagents.

In the latter cases the mesh pores are full, i.e. not accessible any more for molecules of a certain hydrodynamic radius Rₕ₁, due to the swelling behavior of the cross-linked polymers and the resultant mesh in the selected solvent.

In each particular case the potential swelling behavior can be estimated from the available polymer literature. Thus the degree of pore filling can be realized, adjusted and controlled by the selection of the appropriate solvent and pH.

Appropriate solvent means a solvent which is capable to swell the polymeric mesh to an intended degree, according to the rules of polymer solvation, as known to a skilled person. For details see H.-G. Elias, Makromoleküle, Hüthig & Wepf, Basel, Bd.1 (1990), p. 145-207.

With composite materials of the present invention iSEC is the method to determine pore volumes and pore volume fractions.

Protocols as mercury intrusion or BET-nitrogen adsorption, as used for rigid porous materials are not applicable here, because the mesh will collapse after drying.

The following are therefore preferred embodiments for the synthesis and use of composites comprising both adsorptive and non-adsorptive polymers.

In a preferred embodiment, in combination with any of the below embodiments, a functional polymer is introduced into the support material in a shrunk state. A basic polymer, e.g. comprising amino groups, is preferably applied in a solution between pH 8.5 and 13, an acidic polymer, e.g. comprising carboxylic groups, preferably between pH 1 and 6, thus allowing a maximal density of the dissolved polymer under the conditions of pore filling.

After cross-linking and swelling the basic polymer at a pH below 8, respectively the acidic polymer above 6, the space occupied by the polymeric mesh inside the initial support pore volume will increase and finally be maximized by further shifting the pH.

In a preferred embodiment, in combination with any of the below embodiments, one major object of the present invention is reached by the reaction of at least one shrunk cross-linkable polymer with at least one cross-linker, thus forming a mesh, which is selectively swollen or shrunk in certain solvents or buffers.

Under the conditions of characterisation and use the degree of pore filling can be adjusted to a desired level by choosing appropriate solvents or solvent mixtures. By definition, the pores of a polymeric mesh are considered full, if a standard molecule with a selected and well defined hydrodynamic radius Rₕ₁ cannot enter the mesh pores any more. In the present invention, this degree of swelling is calibrated and adapted using the methods of inverse Size Exclusion Chromatography (iSEC) as outlined in the section

Methods and further controlled during the purification process, while maintaining the corresponding swelling state by the presence of the selected buffers. Rₕ₁ is defined as the “size exclusion limit” and is ranging between 1 nm and 20 nm, preferably between 3 nm and 10 nm, most preferred between 4 nm and 6 nm.

Basically the steric exclusion of molecules with a defined minimum (“or critical”) hydrodynamic radius takes place from a particular pore volume fraction, as demonstrated by comparison with the pullulane molecular mass standards, used as model target compounds.

Therefore, in a preferred embodiment, and in combination with any of the above or below embodiments, the degree of polymer swelling is determined by inverse Size Exclusion Chromatography, utilizing a selection of polymer standards of well-defined molecular mass and relating calculated molecular size for calibration and concomitant adjustment of the polymeric mesh by adding the appropriate solvents or solvent mixtures.

According to the present invention, the accessible mesh pore volume increases under swelling conditions and decreases under shrinking conditions in appropriate solvents.

The mesh pore size volume and the mesh size distribution are always related to the space inside or between the particular connected polymer coils or globules, and not to the space initially available or finally remaining in the support material.

The following description is relating to the fundamentals of the composite adsorbent design.

Design element A: Immobilisation of adsorptive polymers in the pores of a support material, whereas only a minor portion of said polymers will be attached to the exterior surface.

The preferred strategy in order to enable the purification of a target compound via complete depletion of any impurities is comprising the provisioning of at least two adsorptive polymers, each of them exhibiting a high affinity towards at least one, preferably a couple of impurities. This couple is comprising a fraction of the chemically related substances among the various structures of the feed impurity inventory. When these polymers are immobilized to at least one support material, preferably the target compounds are not penetrating the resultant polymeric mesh, more preferably are not penetrating the whole composite pore volume and are thus sterically excluded (FIG. 3). When at least two (different) adsorptive polymers 1, 2, . . . n are applied, they are either comprised within the same composite material (Category A 1), or they are incorporated in at least two composite materials based on the same or on different support materials (Category A 2).

When at least two composite materials are concerned, each of them may be furnished with at least one adsorptive polymer.

Each of these polymers may be derivatized in advance or after the immobilization.

Design element B: Immobilisation of at least one inert polymer n+1, n+2 . . . n+i, which is not binding the target compound under the conditions of a separation step, on the boundary surface of the lastly immobilized layer, which is comprising binding polymers. Polymers which are not binding the target compound under the conditions of application are designated inert or non-adsorptive or non-adsorbing.

The last binding or adsorptive polymeric layer means the layer forming the interface with the liquid phase before the inert polymer is immobilized. This upper layer is preferably immobilized within the penultimate/previous step of the manufacturing process.

In one preferred embodiment, in combination with any of the above or below embodiments, the layers comprising binding polymers are completely filling the support pores (details are given in the section Morphology) under the conditions of the synthesis. In this case the inert polymer cannot penetrate the pores of the precursor composite and is thus attached to the exterior surface of said precursor composite, which may be already coated with thin layers of the binding polymers, too (FIG. 3).

In another preferred embodiment, in combination with any of the above or below embodiments, the binding polymers are not completely filling the support pores under the conditions of the synthesis. In this case the inert polymer has access to the pores of the precursor composite and is thus deposited on the boundary surface/top of the last binding polymer (FIG. 4).

Within the present invention a general strategy was worked out to avoid the binding of the target compound to the accessible outer surface of the polymeric mesh of the adsorbent, relying on the shielding of said surface with ligands not capable of significant binding interaction with a target compound under the conditions of separation, in particular in presence of the chosen solvents or buffers.

B 1: This object is preferably achieved attaching or binding or synthesizing at least one inert polymeric layer n+i (i=1,2, . . . k) B 1.1—on the outer surface of a composite which is already comprising the at least one adsorptive polymer,

or of

B 1.2—on the outer surface of a support material itself;

or

B 1.3—on the total accessible surface of said composite, comprising the outer surface as well as the intra-particle surface, i.e., the accessible inner surface of the pore system.

Synthesizing in the context of the present application means the polymer generation from monomers or preferably the derivatisation of a functional polymer before or after its immobilisation.

Alternatively a final layer may be installed, which repels the target compound due to its opposite net charge. Related embodiments, however, are less effective, because a protein with e.g. an isoelectric point above 8 may nevertheless exhibit epitopes or patches with overall negative charges. As a consequence a certain portion of this target compound may be bound to positively charged adsorbents. In particular with low concentrated target proteins this effect will be disadvantageous with respect to the yield.

Examples of non-adsorptive polymers with respect to the interaction with biopolymers in aqueous solution are neutral polar polymers, as listed below.

Preferred examples of non-adsorptive or non adsorbing groups which may be attached via derivatisation to a functional polymer according to section B 2 are listed below. Said principle of non-binding is also applicable in organic solvents, where the polymer on the exterior must be lipophilic, comprising aliphatic and/or aromatic ligands, preferably polystyrene, polypropylene, or copolymers thereof.

In one exceptional embodiment according to B 1.2, namely without applying an adsorptive polymer (number of internal layers is n=0), only the inert external polymer n+1 is attached. This design is advantageous for support materials exhibiting hydrodynamic radii (Rₕ) below or close to the desired exclusion limit of the resultant composite and, at the same time, displaying a high affinity towards the impurities in a feedstock. This is preferably the case with inorganic support materials in both organic and aqueous environment. Preferred for these applications are also support materials comprising commercially available cation and anion exchanger resins, e.g. based on agarose or poly(methacrylate), more preferred are silica gel, alumina, titanium dioxide and zirconium oxides. Alternatively the support pores themselves might be accessible for the target compound, but the non-adsorptive layer does not allow the target permeation. One related example are formyl- or acetyl-derivatives of polyamines, preferably of poly(vinylamine), exhibiting a size exclusion limit Rh above a range between 3 nm and 6 nm.

Preferred pore diameters of the support materials are listed above.

One preferred embodiment, in combination with any of the above or below embodiments, is comprising a porous silica gel covered with an inert, polar neutral polymer, preferably one of the polymers as listed above on the outer surface.

Another preferred embodiment in combination with any of the above or below embodiments is comprising silica gel covered with a polyamine, preferably poly(vinylamine), which is, at least in part, formylated or acetylated. Other preferred embodiments in combination with any of the above or below embodiments are comprising commercially available ion exchangers or mixed-mode media as a support material, wherein the external surface is covered with an inert polymer. Suitable commercial support materials for example are: Capto Q/S/adhere, various Toyopearl®, Fractogel® and Eshmuno® IEX and mixed-mode resins, Q/S/Starax/HEA/PPA/MEP HyperCel® resins, POROS® IEX media, a diversity of Amberchrom® resins, just to list a few prominent examples.

A general method to design and synthesize materials according to paragraph B comprises the adsorption of a high molecular weight neutral polymer or its precursor, excluded from the pores of the support material due to their size, and finally immobilize this polymer layer by either cross-linking or co-valent coupling to one layer of a composite, or directly to the surface of the support material. A precursor polymer, e.g. a polyamine, has then to be further modified by derivatization with an inert group, according to section B2.

Polymer inmmobilization on the outer surface of a support material or composite material.

Among the available methods of polymer immobilisation cross-linking is preferred. The degree of cross-linking for any composite material synthesized for the purpose of the present application. should not exceed 50%. Preferred are 2% to 40%, more preferred 5% to 30%, most preferred are 10% to 15%.

In combination with any of the above or below embodiments, any cross-linker known from prior art is applicable for the immobilization of a polymer according to the present invention.

The cross-linker may either be introduced together with the polymer, in order to allow for a simultaneous reaction of both, or the cross-linking reaction may be carried out separately, in a subsequent step.

It is generally difficult to predict/select an appropriate cross-linker concentration, because only 5% to about 40% of the functional groups should be cross-linked, something which may turn out hard to control. Thus it is not recommended to use excess cross-linking reagent, because a certain amount will also enter the pore volume, if it is full of a good solvent for the cross-linker.

The preferred solution to overcome this drawback is, therefore, to use a two phase solvent system as outlined in detail in the section relating to the category III.c below, wherein the cross-linker is prevented from penetrating the intra-porous volume. In said two phase solvent systems cross-linkers soluble in organic solvents are preferred, like chlorides of dicarboxylic acids, or of other activated, at least bi-valent acids, whereas the internal particle volume is filled with an aqueous solvent. More preferred are bis-epoxides, most preferred is hexanediol diglycidylether.

B 2 Derivatisation of the outer composite or mesh surface

After the final binding polymer was immobilized, another preferred embodiment, also in combination with any of the above and below embodiments, is comprising the derivatisation of the remaining accessible surface, preferably the external surface of the composite particle, with ligands which are not binding the target protein under the conditions of contact, not binding in particular in presence of the solvent used for the separation. Remaining accessible surface means the part of surface contacting the feed or other solvents and solutions as applied during separation steps using said composite adsorbents.

Examples of non-adsorbing groups in aqueous solution, which may be attached via covalent derivatisation are polar uncharged ligands, as listed below.

When converting only the external surface a very high degree of derivatisation is desired. Therefore excess reagent is used, preferably a 1.2-fold amount, more preferred twice more than the number of functional groups in the polymer.

In one preferred embodiment, in combination with any of the above or below embodiments, the pores of the composite starting material are filled with an aqueous solvent again, whereas the derivatisation reagent is insoluble in water and offered in a non-water-miscible solvent. In this way it is avoided that a part of the reagent will be lost in the pores of the support material and thus will entirely be available for the intended reaction.

In one preferred embodiment in combination with any of the above or below embodiments, a polyamine, preferably poly(vinylamine) is immobilized on the external surface of the support material by cross-linking and further derivatized thus generating an inert ligand, preferably using acetic anhydride, acetyl chloride or a lactone. Additional detailed embodiments are outlined in sections II) and III) below.

The following are preferred material embodiments comprising and combining the design principles and elements of A and B.

In one preferred embodiment, also in combination with any above and below embodiments, the pores of a support material are filled and the surface is coated with preferably two adsorptive polymers 1 and 2, thus forming one or two layers, whereas one additional non-adsorptive polymer n+1 is finally attached on the boundary surface of the penultimate layer or polymer (FIG. 3), preferably to the external surface of the support material (FIG. 4), thus creating a composite for depletion purposes.

In another embodiment, also in combination with any above and below embodiments, the pores of a support material are filled and the surface is coated with preferably two adsorptive polymers 1 and 2, thus forming one or two layers, whereas no additional non-adsorptive polymer n+1 is attached on the boundary surface of the penultimate layer or polymer, preferably not to the external surface of the support material, thus creating a composite for depletion purposes. Preferably one of these adsorptive polymers is cationic, the other one is anionic.

In another preferred embodiment, also in combination with any above and below embodiments, the pores of a support material are filled and the surface is coated with one adsorptive polymer 1, whereas one non-adsorptive polymer n+1 is finally attached on the boundary surface/top of this penultimate polymer, preferably to the external surface of the support material, thus creating a composite for depletion purposes.

Preferred embodiments, also in combination with any of the above or below embodiments, are relating to composite adsorbents dedicated to the purification of a feedstock, which is containing at least one target compound and at least one impurity, said composite materials are comprising a support material and at least one non-adsorbing/non adsorptive polymeric layer, not binding the at least one target compound under the conditions of application, in particular in the solvents present during the purification steps, whereas the impurities are adsorbed in the internal volume of the support materials. Preferably the non-adsorbing/non adsorptive polymeric layer is attached to the outer surface of the support material only, not permeating the pore volume due to its size.

“Target compound” refers to any substance of value, subject to a purification according to the present invention, comprising a molecular mass above 500 Da, preferably substances comprising at least one amino acid or/and at least one nucleotide or/and monosaccharide unit, more preferably peptides, proteins, glycoproteins, lipoproteins, oligonucleotides, plasmids, vectors, nucleic acids, RNA, DNA, oligosaccharides, polysaccharides, or any other biopolymer, but also microorganisms like viruses, bacteria or cells and fragments thereof.

The protein is preferably an antibody, pegylated antibody or another derivative of an antibody, or an antibody fragment.

Antibody means here any immunoglobulin, of human or other origin, either as recombinant protein from any kind of cell culture or cell free system for protein synthesis, or isolated from biological fluid or tissue.

Further preferred are recombinant proteins, most preferably recombinant antibodies. Biopolymers are compounds once produced by living organisms.

The feed solution comprises mixtures of synthetic or natural origin. Preferably the feed is a fermentation broth, either filtrated (cell culture supernatant) or crude, still containing solids like cells and cell debris.

Further preferred embodiments, also in combination with any of the above or below embodiments, are relating to composite materials for the purification of a feedstock, which is containing at least one target compound and at least one impurity, said composite materials are comprising a support material and at least one adsorptive polymeric layer, in addition to the non-adsorbing/non adsorptive polymeric layer.

Therefore the present application is providing a composite material, comprising a support material and at least one polymeric layer, whereas the at least one polymeric layer is forming a polymeric mesh and is comprising at least one non-adsorbing/non-adsorptive polymer which is contacting/in contact with/the liquid phase and thus the feed with/containing the dissolved target compound without binding said at least one target compound.

In preferred embodiments, also in combination with any above and below embodiments, the at least one non-adsorbing/non-adsorptive polymer is comprising (at least one) polar residue selected from hydroxyl (OH—), diol, methyloxy (—O—CH₃), formyl-, acetyl-, primary or secondary amide, or ethylene oxy-.

Polar neutral groups or residues are characterized in that they are neither forming ionic, hydrogen bridge, and dipolar interactions, nor significant van der Waals, or dispersive interactions with the target compound in aqueous solutions.

In further preferred embodiments, also in combination with any above and below embodiments, the at least one non-adsorbing/non-adsorptive layer is comprising at least one polar polymer or co-polymer selected from poly(vinyl formamide), poly(vinyl acetamide), poly(vinyl pyrrolidone), poly(vinylalcohol), poly(vinylacetate), poly(ethyleneglycol), poly(hydroxyethyl acrylate), poly(hydroxyethyl methacrylate), poly(acrylamide), poly(methacrylamide), amylose, amylopektin, agarose, any kind of hydroxylmethyl celluloses, hydroxyethyl celluloses, hydroxypropyl celluloses, hydroxypropyl methyl cellulose, methylcellulose, or acetylcellulose

In one related preferred embodiment, also in combination with any of the above or below embodiments, the polymeric mesh is excluding the target compound by size.

The respective two basic design options are schematically shown with FIGS. 3 and 4.

The present application is also providing a composite material for the purification of a target compound from a feedstock, which is containing at least one target compound and at least one impurity,

said composite material is comprising a support material and at least one polymeric layer, whereas the at least one polymeric layer is forming a polymeric mesh and is comprising

at least one non-adsorbing/non-adsorptive polymer which is contacting/in contact with/the liquid phase and thus the feed with/containing the dissolved target compound without binding said at least one target compound,

whereas

the composite material is excluding the target compound by size.

The respective design is schematically shown with FIG. 3.

In one preferred embodiment, also in combination with any above and below embodiments, the polymeric mesh is completely filling the support pores under the conditions of application, characterized in that 90% of the pore volume is not accessible for a selected pullulane standard with a hydrodynamic radius Rₕ₁.

In a further preferred embodiment, also in combination with any above and below embodiments, the hydrodynamic radius Rₕᵢ of said pullulane standard is equal to the hydrodynamic radius of the target compound within a size deviation of Rₕᵢ=+/−1.5 nm.

The present application is also providing a composite adsorbent

comprising a porous support material having an average pore size of 2 nm to 5 mm and at least one non-adsorptive layer and at least one adsorptive layer,

whereas the at least one non-adsorptive layer is immobilized to the boundary surface of the at final/last adsorptive layer.

The present application comprises various embodiments of composite materials and of their preparation, with respect to the morphology, mainly the pore size distribution, the size exclusion limit, and the swelling/shrinking properties of the polymers and layers comprised. A technical lore is provided, how to adjust the pore size exclusion limit of said composite material by means of synthesis and equilibration, with regard to the molecular size of a target molecule to be excluded. Accordingly a variety of designed materials is available differing in their porosity on a nanometer level. Pore size exclusion limit is the nanometer value determined on the iSEC pullulane calibration curve, adequate to exclude a particular target molecule to at least 90% from a composite material pores under the conditions of application.

The target molecule is characterized by a hydrodynamic radius Rₕ₁ and the impurity compound is characterized by a hydrodynamic radius Rₕ₂, wherein Rₕ₁>Rₕ₂.

The composite material is comprising a support material and at least one polymeric layer comprising at least one functional polymer.

The individual layers of said composite material are synthesized and the composite material is equilibrated combining the below parameters, features and materials. In this way composite materials with various and variable pore size exclusion limits Rₕᵢ are generated.

The pore size exclusion limit Rₕᵢ of a particular composite material is thus adapted to the hydrodynamic radii Rₕ₁ and Rₕ₂ such that Rₕ₂<Rₕᵢ

and Rₕ₁Rh1>RₕᵢRhi.

According to the way of preparation and the solvent dependent swelling of the polymers, the layers and the resultant mesh, this exclusion limit Rₕᵢ is a variable size. The term Rₕᵢ indicates that a series of different sizes will be obtained as the function of the swelling degree. In contrast, are Rₕ₁ and Rₕ₂ expressing fix distances in a particular application case.

Accordingly is the present application relating to a process for the preparation of a composite material comprising at least one adsorptive polymer, or at least one non-adsorptive polymer, or at least one adsorptive polymer together with at least one non-adsorptive polymer, characterized in that a combination of the following parameters, features and materials is selected and applied during said preparation, thus generating a characteristic pore size distribution and an appropriate size exclusion limit for a target compound.

The relating parameters, features and materials are:

The pore size distribution of the support material.

The structure of the polymer, mainly its chemical constitution, molecular mass, configuration, and conformation.

The concentration of the particular polymer during the synthesis and the immobilized amount of each particular polymer.

The cross-linker used, mainly its length, polarity, and functional groups.

The degree of cross-linkage of the polymeric mesh.

The reaction pathway of polymer immobilization, precipitation, or synthesis.

The solvent, mainly the solvent polarity, used for the dissolution of the particular polymers and cross-linkers applied for the preparation of the polymeric mesh.

The variation of the pH of said solvent used for the preparation and thus the degree of ionization of the acidic and/or basic residues of the polymer.

In preferred embodiments, also in combination with any above and below embodiments, at least the following three parameters, features, and materials are combined and varied:

The concentration of the particular polymer during the synthesis and the immobilized amount of each particular polymer.

The reaction pathway of polymer immobilization, precipitation, or synthesis.

The solvent, mainly the solvent polarity and solvent pH, used for the dissolution of the particular polymers and cross-linkers applied for the preparation of the polymeric mesh.

In further preferred embodiments, also in combination with any above and below embodiments, the following parameters, features, and materials are combined and varied:

The solvent, mainly the solvent polarity, used for the swelling and shrinking of the particular polymeric mesh.

The pH and the concentration of acids, bases, or/and salts comprised in said solvent used for the swelling and shrinking, and thus the degree of ionization of the acidic and/or basic residues of the polymer.

Combining and varying the above parameters, features and materials, in further preferred embodiments, also in combination with any above and below embodiments, the size exclusion limit of a composite material is set to a value comprising a pore size range between 1 nm and 12 nm, as determined using pullulane standards of known molecular mass and known calculated hydrodynamic radius, preferably under the conditions of application.

Furthermore, in preferred embodiments, composite materials are prepared and equilibrated, and subsequently characterized by their pore size exclusion limit in a range between 1 nm and 12 nm, as determined in 20 mM ammonium acetate buffer at a pH between 4 and 9, using inverse size exclusion chromatography (iSEC) of pullulane standards exhibiting a molecular weight between 1,300 Da and 210,000 Da.

These 20 mM buffers, comprising any pH value within a range between 4 and 9, are defining the standard conditions for iSEC calibration purposes. The pH is selected according to the swelling properties of the immobilized polymers.

As target compounds with a hydrodynamic radius Rₕ in the range between 4 nm and 6 nm are preferably to be excluded from at least 90% of the pore volume, the appropriate pullulane molecular masses for tests under the above conditions are ranging from 21.7 kDa to 48.8 kD.

As no method for a sufficiently accurate direct measurement of pore size distributions is available on a nono-scale level with respect to suspended compounds with varying conformation, iSEC is the preferred method for a satisfactory determination of exclusion limits. The pullulane standards are hereby serving as a scale for the calibration of a distinct length, which is narrow enough to prevent the access of a molecule of a certain size (hydrodynamic radius) with respect to the pores in a composite material.

Therefore, the following procedure is usable, in order to identify and adapt the pore size exclusion limit of a composite material. In a first step the composite material is tested under standard conditions with the calibrated pullulane standards, and the exclusion limit is then shifted by variation of the pH, the buffer concentration and the buffer composition, until the targeted value is approximately reached. Then the size exclusion of the target compound in the feed is examined under the same solvent conditions, preferably using the method of dynamic capacity determination. Finally, the pH and buffer concentration may be adjusted again. Preferred liquids are comprising ammonium acetate solutions. For more details see the section Application below.

In one preferred embodiment, also in combination with any above and below embodiments, a system of components is prepared comprising at least one composite material and at least one liquid phase, whereas the at least one composite material is equilibrated with said liquid phase, thus generating a distinct size exclusion limit as determined with pullulane standards characterized by a defined molecular mass.

Preferred are solvent conditions, allowing the exclusion of a distinct pullulane standard compound selected from a molecular mass range between 1,000 Da and 210.000 Da, exhibiting a related hydrodynamic radius Rₕ in the range between 1 nm and 12 nm, from at least 90% of said pore volume.

In another preferred embodiment, also in combination with any above and below embodiments, the composition and the pH of the at least one liquid phase are varied and selected enabling, to at least 90% of the composite pore volume, the exclusion of a pullulane standard with a molecular mass of a defined value chosen from a range between 1,000 Da and 210 kD. For this purpose preferably the buffer concentration is set to a concentration between 20 mM and 50 mM, and the pH is changed in steps of one pH unit between pH 4 and pH 9. Finally, the buffer concentration and the pH may be further optimized in between the two best previous concentration and pH conditions.

In a further preferred embodiment, also in combination with any above and below embodiments the composition and the pH of the at least one liquid phase are varied, until a target compound exhibiting a hydrodynamic radius Rₕ is excluded from at least 90% of the mesh volume.

The preferred starting point of this adaptation is the buffer concentration and pH as obtained for the pullulane standards with the previous runs.

In one preferred embodiment, also in combination with any above and below embodiments, the solvent conditions are adapted, until a target compound exhibiting the hydrodynamic radius (Rₕ+/−1.5 nm) of the excluded reference pullulane standard is also excluded from at least 90% of the mesh pore volume at the same pH, preferably in the same solvent as used for the iSEC pore size exclusion of said reference pullulane.

In one preferred embodiment, also in combination with any above and below embodiments, said composite materials are characterized by their pore size distribution under the conditions of application, wherein the overall pore size distribution and the exclusion limit are determined by inverse size exclusion chromatography (iSEC), using pullulane standards in the buffer of application preferably at a pH, which allows the swelling of the polymeric mesh.

Application means any step in a separation or purification process, wherein the composite adsorbent is contacted with the equilibration or elution buffer or the solvent for the washing, preferably with the feed.

Mixture of at least two composites.

In one preferred embodiment, also in combination with any above and below embodiments, a mixture of at least two adsorbents, comprising at least one composite material, is prepared, whereas at least one of these composite adsorbents is equipped with at least one adsorptive polymer.

At least one of those adsorbents may comprise any kind of adsorber as commercially available or known from the prior art, preferably ion-exchange resins like Sepharose CM, DEAF, S, and Q, or mix-mode resins like Capto Q or Capto S.

In one preferred embodiment, also in combination with any above and below embodiments, at least two composite materials, each of them equipped with at least one adsorptive polymer, and optionally at least one non-binding polymer are mixed, thus yielding an adsorbent mixture of adsorbents for depletion purposes. At least two of these adsorptive polymers are different.

Within additional preferred embodiments, also in combination with any above and below embodiments, the mixture of at least two adsorbents is comprising at least one composite material bearing only at least one non-adsorptive polymer. Examples for this design are given within the present application, e.g. silica gel coated with poly(vinylalcohol) on its external surface.

In a further preferred embodiment, also in combination with any above and below embodiments, said mixtures of adsorbents are used in processes characterized by a convective flow. Examples are chromatographic processes using packed columns, and related processes like expanded bed and fluidized bed separation techniques.

Moreover, in another preferred embodiment, also in combination with any above and below embodiments, said mixtures of adsorbents are used in batch processes, characterized by contacting the adsorbents with the feed, separating the solid and the liquid phase e.g. by centrifugation, and subsequently removing the supernatant.

Accordingly is the present application relating to a mixture of at least two adsorbents, comprising at least one composite material, comprising a support material and at least one polymeric layer, said polymeric layer is equipped with at least one adsorptive polymer, or with at least one non-adsorptive polymer, or with at least one adsorptive polymer together with at least one non-adsorptive polymer.

In one preferred embodiment, also in combination with any above and below embodiments, the composite material or a mixture of at least two adsorbents, comprising at least one composite material is equilibrated in a liquid, preferably an aqueous solvent, more preferably with a buffer in the pH range between 4 and 9, preferably between 6 and 8, thus creating a defined pore size distribution and the related size exclusion limit, as determined with iSEC under the standard conditions as defined in the section Methods, said size exclusion limit is characterized and the iSEC conditions are calibrated using pullulane standards with a distinct molecular mass and the related hydrodynamic radius Rₕ.

In one preferred embodiment, also in combination with any above and below embodiments, in order to allow a sufficient conformation change in the polymer, characterized by swelling or shrinking, preferably at least one layer in one of said composite materials is bearing at least one polymer with either anionic or cationic residues, or both, as listed above. Preferred residues are primary and secondary amine and carboxyl.

Embodiments according to A 1, also applicable in combinations with embodiments comprising Design Elements of B.

One preferred embodiment, in combination with the above and below embodiments, is comprising a composite material, wherein at least two different functional polymers are immobilized on one support material, thus forming a mesh, and

whereas each particular polymer adsorbs at least one distinct impurity or a couple of impurities in the feed, and whereas the target compound is optionally excluded from the polymeric mesh.

Another preferred embodiment, in combination with the above and below embodiments, is comprising a particulate composite material, wherein at least two different functional polymers are immobilized to one support material, thus forming a mesh, and whereas each particular polymer adsorbs at least one distinct impurity or a couple of impurities in the feed, whereas at least one non-binding polymer is attached to the external surface of said composite particle.

Another preferred embodiment, in combination with the above and below embodiments, is comprising a composite material, wherein at least two different functional polymers are immobilized to one support material, and whereas each particular polymer adsorbs at least one distinct impurity or a couple of impurities in the feed, and whereas at least one non-binding polymer is immobilized on the boundary surface of the penultimate adsorptive polymer attached to the composite material.

A.11 Said at least two polymers are either subsequently attached or

A.12 introduced and connected/reacted as a mixture.

The particular polymers immobilized to the support material preferably exist as thin layers in the shrunken state, said layers are swelling to a certain extent in appropriate solvents.

In order to enable this design the pores of a support material are preferably filled with a solution comprising the at least one polymer, and the solvent is partially or completely evaporated. After aspiration of the particles the desired/actual degree of evaporation is controlled and adjusted by weighing the respective composite batch during the solvent removal preferably at reduced pressure.

In further preferred embodiments, in combination with the above and below embodiments, at least two solutions, any of them comprising at least one polymer are subsequently applied and the solvent is, at least in part, evaporated after each filling step before the respective polymer is immobilized.

Immobilization means that the polymer is fixed to the support surface or in the support pore volume and/or outer rim and will not be dissolved anymore in the solvents used for synthesizing, washing, equilibration, cleaning and application of the composite.

Pore filling means that only the pore volume is filled with a solvent or a solution of the reagents, comprising polymers, derivatization reagents, cross-linkers, and other compounds needed for a reaction. For this purpose the pore volume of the support material is determined using iSEC. After adding the appropriate volume of reagent solution to the amount of support material as calculated/chosen for the synthesis, the weight is determined. Accordingly it is possible with each particular immobilization step after evaporation and filling again to determine the average amount of reagent in the composite pores by subsequent weighing, and thus the apparent volume of the solution, which is present in the pores.

Further Elements of Cross-Linking

In one preferred embodiment, in combination with the above and below embodiments, the polymers of any of the above filling steps are preferably cross-linked, either after aspiration of the initial solution, after partial evaporation, e.g., a concentration step, or after the complete evaporation of the solvent.

The cross-linker is preferably added to the polymer solution already before pore filling, when the cross-linking process shall take place in the initial or concentrated solution. Provided that this reaction is performed after evaporation, the dissolved cross-linker is added in a separate step.

Within another preferred embodiment, in combination with the above and below embodiments, the cross-linker solution is introduced into the pores before the particular polymer solution is applied. This cross-linker solution inside the pores is evaporated in part or completely before the particular polymer solution is applied. Subsequently the cross-linker is diffusing into the polymer solution and will react with the functional groups of the polymer.

Within these embodiments a cross-linker is preferred which does not significantly react within a time period below 30 min. under the conditions of mild evaporation, preferably below 40° C., more preferred below 50° C. Preferred cross-linkers are bis-epoxides as listed below.

In one preferred embodiment, in combination with the above and below embodiments, the polymer immobilization is achieved with at least two distinct steps. More details are given below in the section Synthesis.

Layers Made From Polymers

The design of the present invention is favourably realized by evaporation of the solvents after each preliminary polymer immobilization step. As a consequence, the individual polymer or polymer combination are forming a thin layer patched to the surface of a support material. Those individual layers are either connected and fixed by non-covalent, e.g. ionic forces, but preferably by co-valent binding.

It is defined in the context of the present invention that one layer is comprising at least one distinct polymer. One layer thickness may also be equal to certain range of the overall pore diameters of the support, thus occupying/filling a fraction of the pores with small diameter, or even the whole pore volume under the conditions of porosity measurement. The polymer may be either shrunken or swollen for this purpose.

The preparation of a composite material exhibiting a first layer comprising the partial and complete pore filling with one polymer, is outlined in WO (112908P766PC), and incorporated to the present application by reference. The respective embodiments are supporting the above and below explanations.

Although various design possibilities exist for the constitution of a polymeric layer, whose configuration is complementary to certain impurities, the following scheme simplifies the number of choices and the effort to realize the essential parts of the present invention.

Polymer Constitution

In order to bind any substance which can enter a porous mesh of at least one composite material, the adsorptive polymeric layers are preferably exhibiting different structures, whereas either appropriate functional groups or ligands are attached to a polymer or the respective monomer units are already incorporated in a polymer, thus generating the following polarities:
a) At least one polymer is comprising cationic groups and accordingly exhibiting anion exchange properties, e.g. a polyamine.
b) At least one polymer is comprising anionic groups and accordingly exhibiting cation exchange properties, e.g. a polyacrylate.

c) At least one polymer is comprising lipophilic groups and accordingly binding non-polar molecule sites under aqueous solvent conditions, but not in organic solvents, e.g. an N-alkyl or an N-aryl substituted polyamine.
d) At least one polymer is comprising hydrophilic groups, not binding polar substances in polar solvents, but binding said polar substances in unpolar solvents, e.g. poly(vinylalcohol).

Preferred polymers comprising cationic groups are polyamines as listed above. Preferred polymers comprising anionic groups are poly(acrylate), poly(meth)acrylate, poly(styrene sulphonate), poly(vinyl sulphonate), poly(phosphonates), polyphosphates, and their co-polymers.

Polymers comprising lipophilic groups are preferably synthesized from functional polymers as shown in the various embodiments of the present invention. Alternatively co-polymers are applicable comprising lipophilic and polar monomer units.

Preferred polymers comprising hydrophilic groups are preferably the inert polymers as listed above.

Accordingly, the present application is related to a composite material comprising at least one adsorptive layer and at least one non-adsorptive layer, wherein the at least one adsorptive layer is comprising at least one adsorptive polymer, characterized in that each adsorptive polymer comprises either at least one anionic, cationic, lipophilic and hydrophilic residue or combinations of two, three or four different species/kinds of said residues.

Moreover is the present application related to a composite material comprising at least one adsorptive layer and at least one non-adsorptive layer,

wherein at least one adsorptive polymer is comprising at least two different residues selected from either anionic, or cationic, or lipophilic, or hydrophilic residues.

The present application is also related to a composite material comprising at least one adsorptive layer and at least one non-adsorptive layer,

wherein the at least two different residues in at least one adsorptive polymer are comprising cationic and lipophilic residues.

The present application is also related to a composite material comprising at least one adsorptive layer and at least one non-adsorptive layer,

wherein the at least two different residues in at least one adsorptive polymer are comprising anionic and lipophilic residues.

In one preferred embodiment, in combination with the above and below embodiments, at least one polymer exhibiting at least one ligand with one of the structural elements a), b), c), or d) is attached to at least one support material, either subsequently or as a mixture of at least two of the above moieties. Moieties according to the structure of a) and c) may be immobilized subsequently, for example, followed by a mixture of b) and d). Any embodiments comprising combinations of substances with a constitution according to functional elements a), b), c), and d), and the related steps and orders of immobilisation are within the scope of the present invention, hence not limited to the exemplary embodiments listed below.

In one preferred embodiment, in combination with the above and below embodiments, a composite material is comprising a combination of two polymers exhibiting different structures selected from a), b), c), or d), which are either attached subsequently or as a mixture to one support material.

In another preferred embodiment, in combination with the above and below embodiments, a composite material is comprising a combination of three polymers exhibiting different structures selected from a), b), c), or d), which are either attached subsequently or as a mixture to one support material.

In preferred embodiments, in combination with the above and below embodiments composite materials are comprising at least two particular polymers according to structures of a), b), c), or d), attached to one, two or more support materials.

In another preferred embodiment, in combination with the above and below embodiments, combinations of these structural elements cationic, anionic, lipophilic, and hydrophilic are incorporated to the same polymer, e.g., combining the lipophilic properties according to c) with the ionic properties of a) or b).

Moreover, ligands of various structures can be bound to one particular functional polymer. Thus, it is possible to combine at least two of the functional elements a), b), c), or d) within the same polymer, e. g., derivatizing a polyamine, preferably poly(vinylamine) with an anhydride of a dicarboxylic acid, preferably with succinic anhydride, thus generating a zwitterionic structure comprising amino and carboxylic groups, and optionally with benzoic anhydride, in addition, thus introducing lipophilic groups. The derivatisation with phthalic anhydride and other aromatic or aliphatic or araliphatic anhydrides allows the simultaneous introduction of anionic and lipophilic residues. Preferred are also succinic and glutaric anhydride.

In one preferred embodiment, also in combination with any above and below embodiments, a polymer or co-polymer is comprising maleic anhydride units. After reaction with a nucleophilic compound a bivalent product is obtained, comprising anionic ligands (type b) and hydroxyl (type d) after the reaction with water, respectively lipophilic ligands (type c), if the reagent is, e.g., an alkyl or aryl amine, preferably dissolved in an aprotic solvent for synthesis purposes.

For more detailed embodiments relating to these anhydride containing polymers see sections I and II below.

Additional embodiments are given there, showing how this multivalent ligand design is realized with various reagents, using preferably poly(maleic anhydride), poly(acrylates), poly(vinylalcohol), or polyamines as starting materials.

In one preferred embodiment, in combination with the above and below embodiments, a derivatisation of a polymer with residues comprising a structure according to a), b), c), or d) is carried out in advance of the polymer immobilisation.

In another preferred embodiment, in combination with the above and below embodiments, a derivatisation of a polymer with residues comprising a structure according to a), b), c), or d) is carried out in a solid phase synthesis after the polymer immobilisation.

In one preferred embodiment, in combination with the above and below embodiments, and related to utilization in aqueous solvent systems, preferably up to three polymers of the constitution a), b), and c) are installed in at least one support material, allowing the adsorption of impurity structures complementary to the respective polymer configuration. For the use in unpolar organic solvents up to three layers of the constitution a), b), and d) are installed to at least one support material for said purpose.

Basically the sequence order of attaching the individual layers may be freely chosen. With respect to the impurity composition of the feedstock, more preferred combinations and their synthesis are described in detail below.

Within additional preferred embodiments, in combination with the above and below embodiments, at least one of the residues according to the constitution a), b), c), or d) is already comprised in a polymeric starting material. Examples are polyamines, comprising a type a) amino group, or co-polymers of maleic anhydride comprising aliphatic type c) monomer units.

### DETAILED DESCRIPTION

Categorization by Molecular Properties

For the purpose of categorization the various embodiments of the present invention it is helpful to define the properties of both receptor (composite material) and substrate by means of their size, charge, and polarity:

The chemical nature of each particular polymer comprised in a layer in the pores of the novel composite material is preferably chosen or designed complementary to the structures of a particular impurity, couple of impurities or class of impurities, as defined according to the properties, as listed above and below. Complementary means that the functional groups, ligands, binding sites, or epitopes of a pair of substances are attracting each other due to their constitution, configuration and/or conformation. One attracting substance is preferably an adsorptive polymer, the other substance is a particular impurity or class of impurities.

Examples for pairs of substances with complementary sites are: Receptor-substrate, enzyme-substrate, adsorbent and its ligands-impurity. The most important complementary ligands are comprising ionic, hydrophilic, and lipophilic groups, epitopes are comprising a set of such ligands. The related binding interactions are mainly based on dispersive, van der Waals, dipole, hydrogen bridge, and ionic forces, and combinations thereof.

Class of impurities, chemically related compounds, substances, sites or materials are terms as defined above.

For the purpose of the present invention the substance members in a mixture of impurities are categorized by means of their
Molecular size, defined by the hydrodynamic radius Ri,
Isoelectric point pl, provided that they comprise ionic residues and
polarity (according to their solubility in water, aqueous, and organic solvents), e.g. defined by the log P value or, in the case of macromolecules, simply by the number and density of lipophilic residues in the potential contact area with the polymer layer.

Synthesis of composite according to the design principles and the lore of present application.

The general design of a polymeric mesh/a composite material comprising the constitution as outlined in the above sections A and B and moreover according to the structural categories a), b), c), and d), can be realized by using basically any adsorptive polymers for the synthesis, preferably functional polymers, immobilized using various ways of fixation, inclusive simple precipitation, as long as the polymer is insoluble in the respectively employed solvents.

While the design and structures of the composites in the present invention are novel, beyond the prior art, many of the starting materials, reagents, and building blocks and most of the synthetic steps are known and therefore can be used for the purposes of the present invention in various combinations, as long they are compatible with the abovementioned design principles of the present invention.

Prior Art

Any synthesis steps within the present patent application may be carried out according to the various methods and protocols as known from the prior art. Any chemistry known to a skilled person in the art may be used to realize these strategies. Activation and derivatisation reactions are closely related to the concepts as used in peptide synthesis. The methods, substances, and reactions as e.g. published in Houben-Weyl, Vol. E 22a, 4^{th} Edition Supplement are applicable in many respects. Mainly the chapters carbodiimides, active esters carbonyldiimidazole, and mixed anhydrides are useful.

Without any limitation of other suitable and accessible sources, the following citations are containing useful protocols for polymer immobilization and derivatization, also comprising the chemistry of functional group activation: WO 90/14886, WO 98/32790, WO 96/09116, EP 1 224 975, and Journal of Chromatography, 587 (1991) 271-275.

Substances and Synthesis

According to the present invention at least two different kinds of affinity are created/generated towards a mixture of dissolved or suspended molecules/substances, at least one kind of affinity inside the pores according to design A and a final one on the surface of the penultimate polymer or on the external surface of a support material itself according to design B, and as described within the embodiments for the synthesis below, and in combination with any above and below embodiments.

The term different kind of affinity is defined above. Ionic ligands, binding impurities of different net charge as expressed by their isoelectric point are one very common example. For various more detailed embodiments see below.

Embodiments according to the design elements of section B above are called inert or non-adsorbing and characterized in that their affinity towards the target compounds dissolved or dispersed in a given solvent is very low, preferably with a partitioning coefficient P as defined above, in the particular solvent or buffer used for their application.

For the attachment of polymers, either with interactive or with non-binding properties, three main strategies for the synthesis are available, in order to obtain structures, exhibiting these different affinities and the related selectivities:
I.) One family/set of synthesis embodiments, also in combination with each of the above and below embodiments, is comprising the subsequent or simultaneous immobilization of at least one dissolved/soluble adsorptive, preferably functional polymer (1 or 2 or 3, or . . . n polymers) inside the pores, and optionally at least an inert one (n+1 or n+2 . . . or n+i polymers) attached either to the external support surface or on the boundary surface of the last adsorptive layer, and connected with at least one of the internal polymers. Said immobilization is achieved by the following means: Spontaneous adsorption to the support surface, or precipitation after the evaporation of the solvent, alternatively precipitation after dilution with a poor solvent, or introduction by filling the support pores, each procedure preferably followed by cross-linking inside the support pores. Each of these polymers may be derivatized in advance or after the immobilization according to the principles of section II) or III).

At least two of said polymers (1 or 2 or 3, or . . . n polymers), as well as (n+1 or n+2 . . . or n+i polymers) are different.

Different polymer means that either the structure is different and/or the molecular mass of the respective polymer. Structure is comprising the chemistry, e.g. the copolymer composition or the degree of substitution, but also a different conformation of the polymer, e.g. coil, globule, or any intermediate state between them, is among this definition.

Preferred is the synthesis of composites with three layers, comprising polymers 1, 2, n+1, more preferred are composites with two layers, comprising polymers1, n+1.

Immobilization means that the polymer is fixed to the support surface or in the support pore volume and/or outer rim as defined above. Usually this requested stability cannot be achieved solely by adsorption. Even when a precipitated polymer is not soluble in the solvents of use, there are severe regulatory constrains for the application of such materials, due to potential leaching or degradation. Therefore the polymers are preferably fixed by cross-linking. Another option for the polymer 1 is the covalent binding to the support material, or to the previously attached polymer, according to one of the various methods described in the prior art.

In some cases it may be advantageous to attach a second layer of the same polymer structure to the external surface of the support material, e.g. to achieve an enhanced capacity, the second polymer preferably with a high molecular mass and thus with a greater hydrodynamic radius Rh, whereas the exclusion limit value of the swollen first layer polymer 1 prevents the penetration of polymer 2 into the pores. In general, exploiting the principle of molecular size exclusion, in order to prevent a dissolved polymer of given molecular size from penetration into pores of an appropriately smaller molecular exclusion limit, provides a useful strategy to achieve explicit immobilization of said polymer on the outer surface of a porous composite support material. Therefore, in preferred embodiments, in combination with the above and below embodiments, the pore volume and the exclusion limit of a porous material, either a support or already a composite, are determined using iSEC as described in the section Methods, and an appropriate polymer is selected according to iSEC data, gathered under the solvent and temperature conditions of the synthesis, which does not penetrate at least 70% of said pore volume, preferably 80%, more preferred 90%.

In one preferred embodiment, in combination with the above and below embodiments, a functional polymer, preferably a polyamine, more preferred poly(vinylamine) with an average molecular mass of 40,000 Da together with a cross-linker, preferably of the oxirane-type is filled into the pores of a support material with a nominal pore size of 25 nm and allowed to react at a temperature between preferably 50° C. and 150° C. for a time preferably between 5 minutes to 24 hours. Subsequently a poly(vinylamine) with an average molecular mass of 90,000 Da is immobilized using the un-reacted oxirane groups which serve as an anchor on the particle surface.

In a related preferred embodiment, in combination with the above and below embodiments, the amino groups of the first stage are derivatized before the second polymer is attached, under conditions preventing from hydrolysis of the residual (excess) oxirane groups.
II.) Another set of synthesis embodiments, also in combination with each of the above and below embodiments, is comprising the derivatisation of at least one immobilized functional polymer 1, which is present in the pores, before optionally the next functional polymer 2 and an external polymer n+1 are immobilized.
III.) A third set/family of synthesis embodiments, also in combination with each of the above and below embodiments, is comprising the selective derivatisation of either
   III a.) the functional groups of a particular polymer largely located inside the composite pores, or
   III b.) the functional groups of polymer chains located at the outer ply of the support material or protruding from or beyond the external surface into a liquid, or

III c.) the derivatisation of at least two polymers, e. g., 1 and 2 , respectively, 1 and n+1, with at least two different ligands.

Provided that the ligands attached to the external rim according to synthesis route III b do not exhibit affinity towards the target protein in the solvent of use/application, there is usually no need for the introduction of another non-adsorptive polymer layer according to the design principle B). Accordingly one layer, preferably one adsorptive polymer will be sufficient, if it is selectively derivatized with a non-adsorbing ligand on the outer surface contacting the feed solution. Related embodiments, e.g., with acetylation of a polyamine are shown below.

The following are preferred embodiments relating to the synthesis strategies of set I.) and set II.):

Subsequent or simultaneous immobilisation of at least one polymer inside the pores and one external polymer n+1, optionally combined with a derivatisation of functional or active groups.

These embodiments include several combinations of functional polymers which can be attached to one or more support materials. Numerous additional combinations are possible to create further embodiments according to the design principles and rules, as provided by the present application, as established above and below.

A functional polymer is comprising at least one ligand, residue, or monomer unit, capable to react with a reagent, preferably a nucleophilic or electrophilic one, in particular with a cross-linker. Preferred examples are listed above. Said reagent may become activated in advance, e.g., according to methods known from peptide chemistry, or may be already active, e.g., bearing functional groups like oxirane, anhydride, or acid chloride.

The order of polymer introduction during the synthesis is arbitrary.

The attachment order of the polymers and/or layers can be changed, favourably with respect to the constitution of the average impurity fractions in the composition of the feed.

In one embodiment, together with any of the above or below embodiments, the first polymeric layer on the inner and outer walls of the support material or on the bottom of its pores is selected with respect to high affinity for binding of impurity compounds or the class of compounds with the highest concentration in the feedstock. The related class of impurities will not be adsorbed by the subsequently immobilized polymers, because these are chosen from the group featuring a comparatively small or very small (even close to zero) partitioning coefficient.

In this case a falling concentration gradient of bonded impurities, top to bottom of the pores, or in downward direction along the pore walls, is generated, thus avoiding rapid plugging of pores by highly concentrated impurities.

The layers are inter-connected with each other either by covalent bonds or non-covalent interactions.

The porosity is adjusted according to the desired exclusion limit, varying the parameters already discussed before.

At least the porosity of the penultimate layer n is adjusted to an exclusion limit small enough to prevent the target molecules from access to the pore volume.

The final layer comprising at least one polymer n+1 is either attached to the boundary surface of the previous polymer (FIG. 3) or only to the external rim of the support material (FIG. 4), preferably without penetrating the mesh pores. The appropriate exclusion limits are estimated following iSEC analysis.

In the most preferred embodiment, in combination with any of the above and below embodiments, this final layer exhibits a polarity and/or charge without affinity towards the target compound, in the solvent used for the purification of a feed, comprising the at least one impurity and at least one target compound. Poly(vinylalcohol) (e.g. Poval, Exceval, brands of Kuraray, Hoechst, Germany) is the preferred choice of a non-adsorptive polymer. Other non-adsorptive polymers are listed above.

Provided that the final layer at the interface between stationary phase and mobile phase, preferably at the outer rim of the particle, comprises an amino group containing polymer, negatively charged high molecular mass impurities, e.g., nucleic acids, are expected bind to this external surface, thus covering the entire outer surface and hence hindering in many cases the adsorption of target molecules. At the same time depletion of nucleic acids is accomplished, something that is to be considered a big advantage when treating fermentation broth comprising at least one target protein. However, target proteins owing an isoelectric point (p1) below 7 may be bound to a certain extent on the external surface of the above described cationic polymer structure, which of course does not interact with basic proteinsdue to electrostatic repulsion. For this reason the external layer should preferably be designed (electrostatically) neutral, because otherwise acidic or basic proteins, as far as targeted as products, might become bound, due to their overall negative or positive charge, under the operational conditions. However, if mixtures of at least two composite materials are applied together in a single unit operation step of downstream processing a fermentation broth, one of them should preferably be positively charged on the external surface, in order to bind nucleic acids and oligonucleotides. The concentration of said positively charged composite is then chosen at a minimum amount, just sufficient to bind these impurities. The optimal ratio of both composites is established after the measurement of the nucleotide concentration of the feed and the nucleic acid binding capacity of the adsorbent bearing amino groups on the outer surface, which is in the range of 1 mg to 1.5 mg DNA (from calf thymus) per ml. The exterior surface of the at least one additional/other composite is kept neutral. This is the appropriate strategy to keep negatively charged target proteins as solutes, thereby avoiding respective losses, while simultaneously binding oligonucleotids to the composite material.

The following are preferred embodiments of the present invention for the synthesis of composite materials with two (1, 2) or three adsorptive polymers (1, 2, 3) and one inert polymer n+1.

In one embodiment, in combination with any of the above and below embodiments, the polymer 1 attached first to the porous support material is lipophilic, comprising a co-polymer exhibiting aliphatic or aromatic monomer units, together with polar monomer units, preferably comprising poly(vinylalcohol)-co-polyethylene, or co-polystyrene. Preferred are also functional polymers derivatized with an aliphatic or aromatic residue. More preferred are derivatized polyamines, as exemplarily outlined with the embodiments above and below.

In aqueous media the lipophilic residues or sites inside the pore exhibit sufficiently strong affinity towards impurities of various structures, thus enabling appararently quantitative binding, resulting in extensive depletion, of these solutes.

In one embodiment, in combination with any of the above and below embodiments, the second polymer attached to said lipophilic polymer is comprising a polymer with cation exchanger properties. Preferred polymers containing acidic groups are listed above. Additional preferred anionic polymers are hydrolysed co-polymers of maleic anhydride.

Alternatively an appropriately modified polymer is applicable comprising anionic residues or ligands bound to any functional polymer, e.g. a polyamine derivatized with succinic acid residues (Example). These anionic polymers are capable of adsorbing basic impurities, preferably those with pl values above 7.

In one embodiment, in combination with any of the above and below embodiments, a third layer comprises an anion exchanger polymer, preferably a bare polyamine attached to the cation exchanger ligands of polymer 2.

In one embodiment, in combination with any of the above and below embodiments, said third layer (layer 3) comprises at least one non-adsorbing polymer n+1, n+2, n+i.

Preferred embodiments, in combination with the above and below embodiments, are combining lipophilic and ionic polymers, either cationic or/and anionic. Also preferred are solely cationic and/or anionic polymers inside the pores, whereas the external surface is shielded with a hydrophilic polymer.

Also preferred are embodiments combining lipophilic, anionic and cationic polymers inside the pores, whereas the external surface is shielded with an inert polymer.

Accordingly the present invention relates to a composite material and the synthesis of said composite materialcomprising a support material and a combination of maximal four functional polymers, whereas up to three functional polymers are immobilized mainly inside the pores of said support material, whereas one functional polymer is bearing lipophilic monomer units, residues or ligands, one functional polymer is bearing cationic monomer units, residues or ligands, one functional polymer is bearing anionic monomer units, residues or ligands, the functional polymer finally attached to the penultimate polymer, alternatively to the external surface of the support material is bearing weakly hydrophilic groups, non-adsorbing for bio-polymers in aqueous solutions, preferably hydroxyl-, acetyl-, formyl-, amide, or oxymethylene groups.

Mainly inside means that a thin, at least mono-molecular layer of the particular polymer may also be immobilized to the external particle surface.

In one preferred embodiment, in combination with the above and below embodiments, the first layer is comprising an amino group containing polymer or co-polymer and the inert polymer n+1 is preferably one of the polymers listed above.

Embodiments comprising poly(maleic anhydride) and derivatives thereof.

In one preferred embodiment, also in combination with any of the above and below embodiments, a polymer or co-polymer is comprising maleic anhydride units. After reaction with a nucleophilic compound a bivalent product is generated, comprising anionic ligands and hydroxyl (groups) when reacted with water, respectively carboxyl groups together with lipophilic ester or amide groups, when the reagent is, e.g., an aryl- or alkyl alcohol, or an amine, preferably dissolved and reacted in an aprotic solvent.

Accordingly is the present application related to a composite material, wherein at least one adsorptive polymer is comprising poly(maleic anhydride) building blocks/monomer units, which are comprising in turn precursor ligands for anionic and lipophilic or hydrophilic residues.

The present application is also related to a composite material, wherein the at least one adsorptive polymer is comprising hydrolysed poly(maleic anhydride) monomer units, comprising anionic and lipophilic or anionic and hydrophilic residues.

In one preferred embodiment, in combination with the above and below embodiments, the first layer is comprising a polyamine, preferably dissolved in aqueous solvent, optionally together with a cross-linker. After cross-linking and evaporation of water and optional organic solvents, the resultant composite is preferably dried. Subsequently a dissolved maleic anhydride polymer or alternating copolymer, preferably poly(isobutylene-alt-maleic anhydride) or poly(ethylene-alt-maleic anhydride), is added, whereas pores and the interstitial volume between the particles may be filled with the reaction solution to a certain extent, preferably between 60% and 120% of the support sedimentation volume.

This intermediate polyamine composite is reacted with the maleic anhydride polymer at temperatures preferably between 20° C. and 120° C. over a time period between 30 minutes and 24 hours. The two polymers are connected via amide bonds and salt bridges, thus forming two layers, whereas anhydride groups remain intact for potentially desired further chemical modifications, i.e., ring opening reactions, esterification, amidation and other known typical carbonyl chemistry.

In one preferred embodiment, in combination with the above and below embodiments, the boundary surface of the maleic anhydride layer or the external surface of the above composite are finally covered by a high molecular weight inert polymer n+1, preferably with one of the polymers listed above, and preferably allowing available anhydride groups of the poly(maleic anhydride), protruding into the external particle space, to react with the nucleophilic residues of the n+1 polymer.

In one embodiment, in combination with the above and below embodiments, the maleic anhydride polymer is additionally cross-linked using a defined amount of bi- or multivalent nucleophilic reagent, preferably an aliphatic diamine.

In one preferred embodiment, in combination with the above and below embodiments, after the coupling of at least one polymer with the maleic anhydride polymer the residual anhydride residues are converted into carboxyl groups together with hydroxyl-, or ester- or preferably amide groups, preferably with nucleophilic compounds like hydroxyl OH— or alcohols, more preferred with amines. Most preferred is aminoethanol as the reagent of choice if the final product should not become lipophilic. The hydrolysis is preferably performed in aqueous solvents. The ester and amide formation are accomplished in aprotic organic solvents, preferably polar to medium-polar ones, more preferred DMF, dioxane, THF, methyl t-butyl ether (MTBE), dibutylether, dichloromethane, or toluene.

In one preferred embodiment, in combination with the above and below embodiments, the first layer is comprising a polymer or copolymer containing maleic anhydride units, preferably poly(isobutylene-alt-maleic anhydride) or poly(ethylene-alt-maleic anhydride), and after evaporation of the solvent, a polyamine is introduced, preferably dissolved in water and optionally together with a cross-linker, the resultant intermediate composite is preferably aspirated, and the compounds are reacted at temperatures preferably between 20° C. and 120° C. for 30 minutes to 24 hours. The residual anhydride residues are finally converted into carboxyl groups together with hydroxyl, ester or preferably amide residues, preferably by reaction with modestly nucleophilic compounds like polyols, or primary or secondary alcohols, more preferred with amines.

In one preferred embodiment, in combination with the above and below embodiments, the amino polymer and the nucleophilic compound are added simultaneously.

In one embodiment, in combination with the above and below embodiments, the maleic anhydride polymer is cross-linked prior to the addition of the aqueous polyamine solution, preferably using a defined amount of bi- or multivalent nucleophilic reagent, preferably a diol or a diamine, more preferably an aliphatic or aromatic diamine. Most preferred are ethylenediamine, propylenediamine and 1,4 bis (amininomethyl)benzene.

In an additional preferred embodiment, in combination with the above and below embodiments, the external surface is finally covered with a high molecular weight inert polymer as listed above, and preferably immobilized by reaction with residual oxirane groups of the cross-linker.

In one preferred embodiment, in combination with the above and below embodiments, the maleic anhydride units of a polymer or a related co-polymer are allowed to react before or after immobilization with a defined amount of a nucleophilic substance, preferably an alcohol or amine, bearing a lipophilic or a hydrophilic residue, more preferably with a residue formally comprising an aliphatic or aromatic radical, most preferred with aminoethanol, phenylethylamine, benzylamine, or hexylamine. The remaining anhydride functions are sufficient for the final immobilisation step.

In combination with the above and below embodiments the polyamine is immobilized only via ionic forces between the carboxylic groups of the first polymer, e.g, after the hydrolysis of the maleic anhydride residues containing polymer, or directly after contacting with an acidic polymer. Preferred polymers containing acidic groups are listed above.

The amino polymer may finally be cross-linked.

Optionally, the non adsorbing external polymer is attached. Preferred non adsorbing/non-adsorptive polymers are listed above.

In a further preferred embodiment, in combination with the above and below embodiments, the composite obtained after immobilization of a polymer or copolymer containing maleic anhydride and after its derivatisation with a nucleophilic substance, is directly covered with a layer of an inert polymer, as defined/listed above.

It is also possible within another preferred embodiment, in combination with the above and below embodiments, to react the composite comprising a polymer or copolymer containing maleic anhydride units with a polyamine of high molecular weight, thus not capable to enter the composite pores, preferably with poly(vinylamine) of 90,000 Da or a co-polymer thereof, thus generating carboxylic groups inside the pores and a cationic layer on the outer surface.

Within another embodiment, the maleic anhydride containing polymer is filling the complete pore volume, whereas an inert nucleophilic polymer is attached to the external surface.

In one further preferred embodiment, in combination with the above and below embodiments, the first polymer attached is containing acidic groups, the next polymer basic groups, and the final polymer is inert.

Preferred polymers containing acidic groups are listed above.

Basically the order of polymer attachment may be changed within various additional embodiments, also in combination with the above and below embodiments.

Preferred embodiments obtained by derivatisation relating to the categories II.) (non-selective) or III a.) (selective).

The following embodiments are listing several functional polymers which can be attached to one or more support materials and subsequently derivatized. Numerous additional combinations are possible according to the principles and rules as given with the present invention/as established above and according to the comprehensive prior art synthesis methods.

The order of polymer introduction is arbitrary.

In the following preferred embodiments, in combination with any above and below embodiment, general procedures are described for the synthesis of a composite material which is lipophilic inside the pores, but hydrophilic on the particle surfaces.

Lipophilic means that the respective polymer is bearing either aliphatic or aromatic, heterocyclic and/or other hydrocarbon groups at a degree of derivatisation between 2% and 98%, preferably 5% and 80%, most preferred 10% and 50%. Preferred lipophilic ligands or residues are preferably benzoyl-, benzyl-, phenyl-, naphthyl-, short- and long chain alkyl- (n=1 to 20), different kinds of branched alkyl, cyclopentyl-, or cyclohexyl-. When the lipophilic residues are already incorporated to a precast polymer or copolymer, the concentration of lipophilic groups should be within the same range as described above and below for derivatisation of immobilized polymer layers.

In aqueous solvents an interior lipophilic surface will exhibit an enhanced affinity for almost any substances, preferably for proteins, peptides, lipoproteins, lipopolysaccharides and related compounds, which are small enough to enter the pores, whereas, at the same time, an external hydrophilic surface will not bind the target molecules.

It must be avoided, however, to glue lipophilic polymer chains together. For this reason the concentration of the lipophilic ligands must not exceed a critical score, which has to be figured out experimentally.

Strong attractive forces exist between the, e.g., phenyl or alkyl groups introduced, impeding the swelling in aqueous systems. This is why a too high degree of derivatisation will inhibit a potential swelling and thus must be avoided.

It has to be checked by iSEC, that the lipophilic derivative shows sufficient swelling in the application buffer, exhibiting a porosity with Rh values between 0.5 nm and 10 nm, preferably 1 nm and 6 nm, most preferred between 2 nm and 4 nm, determined utilizing iSEC with pullulane calibration molecular weight standards, preferably at the pH, more preferably under the solvent conditions (composition and pH) of application.

In order to establish the optimal porosity, a small series of composites are synthesized with different concentrations of lipophilic units, preferably with 10%, 20%, and 30% of the monomer units in total. The porosity monitoring by iSEC, carried out in the solvent of the desired later application, easily reveals whether the pore size of the resulting mesh is established according to the intended range.

The extent and strength of lipophilic interactions between polymer network and solutes is triggered by the selection of appropriate ligands, e.g., according to the building blocks as listed above.

The desired affinity (binding strength) and pore size distribution are eventually established by adjusting the degree of derivatisation.

Using such ligands or a combination thereof, also supplementary hydrophilic/polar interactions may contribute in a specific way to said affinity.

Such partially lipophilic composites equipped with a cationic exterior/upper layer or surface are to be used to enhance the binding affinity for proteins which can enter the mesh pores, e.g., Host Cell Proteins (HCP) with a molecular weight below 100,000 Da., thus providing improved depletion properties, whereas nucleic acids will still be bound.

In one preferred embodiment the internal amino polymer 1 is containing lipophilic residues, whereas the external polymer 2 is an amino polymer.

As an example, this design is realized in one embodiment using a general procedure for the synthesis of composites with poly(vinylamine) on the exterior rim (layer 2) and a functional polymer 1 with lipophilic, e.g., phenyl groups inside the pores.

In preferred embodiments, in combination with the above and below embodiments, the basic properties of amino-group containing composites are conserved, while lipophilic groups are introduced, preferably using epoxy reagents bearing a lipophilic residue.

Lipophilic Basic Composite Embodiment

In one preferred embodiment, in combination with the above and below embodiments, a composite bearing an amino polymer becomes protonated after equilibration with a monobasic acid or a salt thereof to a pH below 6, preferably using 50 mM hydrochloric acid, more preferred 50 mM ammonium acetate. Accordingly the polymeric mesh is swollen and completely accessible for the derivatisation reagent. This suspension is aspirated and the wet cake is incubated with a concentrated solution of an epoxide, whereas the reagent is equally distributed inside the swollen polymer.

After aspiration the pH is changed to a value above 8 introducing, e.g., about 1 M triethylamine solution, thus deprotonating the ammonium residues of the polymer. Under this condition the reaction with the epoxide starts. Because the reaction velocity with epoxides at room temperature is slow, the reaction should be performed at enhanced temperatures, preferably between 40° C. and 120° C., more preferred between 50° C. and 80° C. over 5 min to 48 hours, preferably for 10 min to 6 hours, more preferred during 20 min to 60 min.

Provided that the particles are dry on the external surface, the major part of the amino groups on this exterior surface remains unreacted, as the epoxide reagent is only available inside the composite pores.

General amide type embodiment comprising composites with different degree of lipophilic derivatisation.

Further preferred embodiments are comprising the internal reaction with 5%, 10%, and 20% equivalents of any anhydride, which is bearing lipophilic groups, like benzoic acid anhydride, preferably in DMF, as described in Examples. As a starting material preferably a poly(vinylamine) containing composite adsorbent is used directly after the pore filling and cross-linking, in general prior to hydrolysis of the remaining epoxy groups, preferably after treatment with an acid, in order to swell the mesh. In this case a second external aminopolymer can be attached by binding to those oxirane groups at 50° C. to 120° C., that have been left over after the cross-linking step. Preferred external polymers are the inert polymers as listed above.

Second Basic Layer Attached to Lipophilic Amide Embodiment

In another preferred embodiment, in combination with the above and below embodiments, the related amide, preferably benzoyl intermediate product is subsequently transferred to 50 mM ammonium acetate again, at preferably a pH between 4 and 6, and a second polyamine layer, preferably poly(vinylamine) with high molecular mass is attached by heating to about 60° C. via the residual epoxy groups left after the pore filling step. Finally the acidic quenching of the oxirane groups is carried out, which have not been reacted with the second polymer.

For impurity depletion purposes the appropriate pH is below 6.5 during contacting the feedstock with the composite adsorbent. During the synthesis of the above embodiment, however, an exposure of more than 10 min at a pH below 4 must be avoided, in order to prevent the hydrolysis of the remaining oxirane groups, if they are required for the subsequent covalent binding of the external layer. Under these conditions the amino groups are protonated and repelling each other, while the amino polymer is swollen, thus preventing the second polymer from access to the pores. As long as the proteins which are captured due to lipophilic interactions are not to be recovered, said swelling requirement remains the only limitation for the degree of derivatisation.

Embodiments using only the capping of remaining epoxy groups after cross-linking.

The most simple approach to obtain a slightly lipophilic inner surface in a porous polymer solely utilizes the remaining epoxide groups of the cross-linker after the first immobilization step. While adjusting conditions, in order to maintain residual un-reacted oxirane groups intact, the second polyamine, preferably poly(vinylamine) layer is attached first to the external surface, as described above. The residual internal epoxide groups are then converted by reaction with an amino compound, an alcohol or another nucleophilic compound, preferably dissolved in DMF or DMF-water or even water at temperatures between 50° C. and 120° C., into their respective derivatives. For this purpose the intermediate composite is kept swollen at a pH below 6.5. After the reagent has been distributed in the pores, the excess reagent solution is preferably aspirated.

Subsequently the reaction mixture is heated for a sufficient time period, which depends mainly on the chosen temperature. Preferred are phenylethylamine, hydroxy benzyl amine and dopamine as ligands.

Embodiments Utilizing an Activated Polymer

In one preferred embodiment, in combination with the above and below embodiments, the immobilized polyamine, poly acid, or polyalcohol may be activated by any known procedure known from the prior art, e.g., using epichlorohydrine under aqueous conditions or chloro carbonic ester, or carbonyl diimidazole (CDI), both in aprotic organic solvents, or carbodiimides either in aqueous or organic solvents. Preferred poly acids are poly(acrylate) and poly(methacrylate). Preferred polyalcohols are poly (vinylalcohol) and Poly(vinylalcohol) co-polymers.

It is advantageous to work with a molar excess of an activation reagent, provided it is bivalent, in order to avoid undesired cross-linking.

The derivatisation with an amine of a molecular weight of at least 100 Da should allow to estimate the reactive epoxy concentration from elemental analysis data (CHN analysis).

Procedures for the selective derivatisation according to design II and III, applying two phase chemistry.

In order to create adsorbents according to the design II or III, the preferable approach comprises synthesis routes using two immiscible solvents.

This technique allows to modify the inner surface of a particle or other porous support material, while leaving the external surface unmodified, and vice versa. For this purpose the pore volume is either filled with an organic solvent, immiscible with water, or with aqueous solvent mixtures. The liquid phase of the suspension is accordingly either aqueous or organic.

Preferred are water or buffered aqueous solutions in combination with ethers, aliphatic and/or aromatic as well as halogenated solvents. More preferred organic solvents are toluene, dichloromethane, trichloroethane, n heptane and 1-octanol. Also esters like ethyl acetate are feasible.

The organic liquid phase is usually a solution containing the required reagents for surface modification. Preferred reagents are electrophilic compounds, not or poorly water soluble, more preferred are activated carboxylic acids, most preferred are anhydrides, carboxylic chlorides and -azides, epoxides, and alkyl halogenides.

The interface between the organic solvent and the aqueous phase can be visualized using the zwitterionic dye Betain 30 in order to control the extent of particle volume filling with the selected solvent. After aspiration of a suspension comprising porous support materials or composites it is possible to slightly shift said interface by controlled slow evaporation of the solvent, left inside the pores. The removal of solvent is preferably determined by continuous weighing of the materials.

In one embodiment, in combination with any of the above and below embodiments, a wet porous support material or composite material is kept between 20° C. and 120° C., in order to remove the targeted amount of solvent from the pores.

Basically any functional polymer is suited for the attachment either inside or outside the pores of the support material. Preferred are polymers with activated or electrophilic or nucleophilic ligands/functional groups, more preferred are polymers containing epoxides, anhydrides, lactone, hydroxyl, amine, or carboxyl groups. Moreover, any kind of functional group may be further activated using reagents as known from the literature.

More preferred are poly-alcohols like poly(vinyl alcohol), poly acids like poly(acrylates) or poly(sulphonates), and poly anhydrides like poly(maleic anhydride) and copolymers thereof. Most preferred are polyamines, in particular poly(vinylamine) and co-polymers thereof, because the primary or secondary amino group is ideal for derivatisation, particularly for the preparation of stabile amides.

Poly(vinyl alcohol) can basically undergo the same reactions with activated compounds, forming esters with acids like carboxylic, sulfonic or phosphonic compounds or ethers with epoxides or alkylhalogenides which are preferred for stability reasons with respect to the product.

Important is a proper solvent exchange to make sure the interface between two immiscible solvents is located nearby the external surface of the particles. For the exchange of the solvents prior to and after a reaction, a stepwise transfer from aqueous to organic conditions is recommended, beginning with ethanol, then acetone and finally a hydrophobic organic solvent, preferably toluene or dichloromethane. This solvent order may be reverted, in order to return, e.g., from toluene to water and then optionally to an aqueous buffer. Five times digesting with five bed volumes per solvent exchange step is considered usually sufficient, provided the liquid is carefully aspirated after each step.

Basically any kind of ligand combinations can be created by the selective reaction of the polymers contained inside the pore volume and on the external surface of particles. Respective chemical reactions are generally known by a skilled person. The following are preferred combinations of polar and non-polar regions in composite materials:

1) Composite which is Inside Lipophilic—Outside Cationic

Preferred embodiments are comprising a functional polymer, more preferred a polyamine, modified by derivatization with aliphatic or aromatic residues, dedicated to modify the interior pore surface, whereas the amino groups on the external rim of a particle remain in original condition, because the aqueous environment on the outer particle surface prevents them from coming into contact with the lipophilic reagents.

This kind of selective reaction, spatially restricted to surfaces in contact with a respectively compatible solvent comprising the first liquid phase, at the same time precluded from taking place in the correspondingly incompatible second liquid phase, where both phases are separated by an interface between immiscible solvents, is also applicable to the above and below embodiments, also in combination with any other embodiment described herein.

In the same way it is possible to generate e. g. ethers from poly(vinylalcohol) and epoxyalkanes.

For more detailed embodiments comprising the combinations 1) to 12) see below.

2) Composite which is Inside Lipophilic—Outside Anionic

Preferred embodiments are comprising a functional polymer, more preferred a polyamine, further modified inside of pores by derivatization with aliphatic or aromatic residues, where reagents are dissolved in organic solvent, and, after solvent exchange, by derivatizationon the outer surface, with an activated carboxylic, sulfonic, or phosphonic acid, preferably an anhydride of a dicarboxylic acid, more preferably with succinic anhydride or glutaric anhydride.

3) Composite which is Inside Lipophilic—Outside Non-Adsorbing Preferably Polar

Preferred embodiments are comprising a functional polymer, more preferred a polyamine inside derivatized with an aliphatic or aromatic residue and subsequently externally with a lactone, acetic anhydride or acetyl chloride.

4) Composite which is Inside Cationic—Outside Anionic

Preferred embodiments are comprising a composite filled with a polyamine, the resin equilibrated to a basic pH, preferably 8-11, preferably with an aqueous base, then aspirated, optionally washed with a solvent not miscible with water, and subsequently derivatized in suspension, by the addition of an anhydride of a dicarboxylic acid, preferably succinic acid, dissolved in a solvent not miscible with water.

5) Composite which is Inside Cationic—Outside Non-Adsorbing, Preferably Polar.

The support material is filled with a polyaminein acidic aqueous solution, whereas the external rim is kept deprotonated in the presence of organic solution of a base, preferably an amine not soluble in water, more preferably octylamine, and derivatized with a reagent generating amide or hydroxyl groups, preferably acetic anhydride, or a lactone dissolved in a non water-miscible solvent.

6) Composite which is Inside Cationic—Outside Lipophilic

The same procedure as above under 4) or 5), but utilizing an anhydride of an aliphatic or aromatic carboxylic acid, preferably with at least four carbon atoms, more preferred benzoic acid anhydride, as the derivatizing reagent.

7) Composite which is Inside Anionic—Outside Cationic

Preferred embodiments are comprising composites entirely surface-coated with a polyamine and with the interior portion of the surface modified by reaction with an anhydride of a dicarboxylic acid, whereas the amino groups located on the outer surface are kept unmodified in original condition.

8) Composite which is Inside Anionic—Outside Non-Adsorbing, Preferably Polar.

Preferred embodiments are comprising composites entirely surface-coated with a polyamine and with the interior portion of the surface modified by reaction, in a first step with an anhydride of a dicarboxylic acid, whereas in a second step the amino groups on the outer surface are modified by reaction with a lactone or with acetic anhydride, dissolved in a solvent immiscible with water, where the second step is carried out after exchanging the solvent inside the pore volume into aqueous solution adjusted to a pH below 6.

9) Composite which is Inside Anionic—Outside Lipophilic

The same procedure as above under 8), but with a hydrophobic reagent chosen for the modification of the outer surface, preferably a lipophilic anhydride or lactone, more preferably benzoic acid anhydride.

10) Composite which is Inside Hydrophilic—Outside Cationic

The polyamine covered composite is modified at the inner surface by reaction with acetic anhydride, or a lactone dissolved in a solvent immiscible with water. During this procedure the particles are exceptionally not suspended in a solvent, thus keeping the amino groups protruding from the outer surface in unmodified condition.

This is a general procedure, in combination with any of the above and below embodiments, applicable for any of the combinations of 1) to 12), avoiding contact between outer particle surface and solvent, thus restricting chemical modification reactions exclusively to internal surfaces, leaving outer surfaces in original condition, which is the general intention of this particular embodiment.

11) Composite which is Inside Hydrophilic—Outside Anionic

The polyamine containing composite is modified on the inner surface by reaction with acetic anhydride or a lactone, dissolved in a solvent immiscible with water. During this step the particles are not suspended in a solvent, thus preventing amino groups protruding from the outer surface from participation in said reaction. Subsequently the pores are filled with water, while the amino groups on the external surface are allowed to react with a chloride or anhydride of a bivalent acid, e.g., sulphonic, phosphonic or carboxylic acid, more preferred maleic, glutamic anhydride, most preferred succinic anhydride, dissolved in a solvent immiscible with water.

12) Composite which is Inside Hydrophilic—Outside Lipophilic

The same procedure as under 11), but the final reagent is a lipophilic anhydride.

The embodiments 1)-8) are preferably used in separations of biologicals, where the feed and the mobile phase are aqueous. The embodiment 3) is particularly suited for reversed phase applications.

The embodiments 10)-12) are preferably used in separations of solutes, where both feed and mobile phase are organic.

Embodiments 6), 9), and 12) serve equally well under/in aqueous and organic conditions solvents.

Structures with the same or related polarity compositions as outlined under 1)-12) are also available using various polymers, reagents, and routes of synthesis, which are known to a skilled person.

Accordingly is the present invention providing a method for the selective derivatisation of the functional groups of a polymer which is located inside a polymeric mesh,

characterized in that the polymeric mesh is filled with a solution of a derivatisation reagent in a solvent which is not miscible with water, whereas

the volume outside of this mesh is filled with an aqueous solvent.

The present invention is also providing a method for the selective derivatisation of the functional groups of a polymer which is located inside a polymeric mesh,

characterized in that the polymeric mesh is filled with a solution of a derivatisation reagent in a solvent which is either aqueous or not miscible with water, whereas

the volume outside of this mesh is not containing a liquid.

Moreover, is the present invention providing a method for the selective derivatisation of the functional groups of a polymer which is located on the boundary surface of a polymeric mesh,

characterized in that the polymeric mesh is filled with an aqueous solvent, whereas the volume outside of this mesh is filled with a solution of a derivatisation reagent in a solvent which is not miscible with water.

Basically a reagent, insoluble in water or aqueous solvent mixtures, may be dissolved and allowed to react with the target polymer in the aqueous phase, while applying the general principles and rules as explained above, whereas no reaction occurs in the organic phase.

The derivatisation reagent is preferably insoluble in water to a vast degree. Preferably the partitioning coefficient log P_{ow} is higher than 1.5, more preferred higher than 2.

The derivatisation reagent may become gradually inactivated to a certain extent, by side reactions, e.g., hydrolysis, taking place at the interface of the solvent—water—system, as long as the main reaction with the functional groups of the polymer proceeds fast enough to achieve the intended degree of derivatisation. Preferably a 1.2 fold excess of derivatisation reagent is applied, more preferred 1.5 mole equivalents.

In order to achieve a complete surface coverage and a high degree of derivatisation, the particular reaction may be repeated once or twice.

The polymeric mesh is hereby either filling the entire pore volume of a porous support material (FIG. 3), or it is only filling a fraction of said pore volume (FIG. 4).

The following protocols for the selective introduction of ligands with different polarity and charge are comprising preferred embodiments of methods applying two immiscible solvents and targeting combinations of polymer derivatives. These examples are only a selection of numerous embodiments, which can be carried out according to the teaching (technical lore) of the present invention. A person skilled in the art will knows, which polymers and which reagents are suitable for the purpose of derivatisation, in addition.

General: It is generally advantageous to start from a swollen polymer conformation before a reagent is introduced, in order to achieve equal distribution of reagents all over and inside the polymer mesh. When polymers with ionisable groups are applied, the following procedures are applicable in order to generate a cationic or anionic surface, in turn giving rise to the polymer to adopt the desired swollen conformation.

The protonation of the amino groups using a monobasic acid or an acidic buffer with pH below 6.5 induces sufficient swelling of the polymer, thus allowing the subsequent equal distribution of the reagent solution inside the mesh. Bivalent or multivalent acids like sulphuric, phosphoric or succinic acid are less suitable for swelling, because they may act as cross-linkers, due to their dual or multiple ionic functionalities.

Acidic polymers, comprising, e.g., carboxylic, sulphonic, or phosphonic residues, are accordingly adopting swollen conformations, following deprotonation under basic conditions, i.e., in the presence of basic solvents.

In this way the composites of the above and below embodiments are treated in advance of reactions inside, but also outside the pores.

Embodiment According to Route 1)

Selective reaction inside the pores comprising a (single) one-pot method for the synthesis of a composite material which is lipophilic inside and cationic on the external surface.

In one preferred embodiment, in combination with the above and below embodiments, the pores of a precursor composite material filled with a mesh of cross-linked poly(vinylamine), e.g., synthesized according to Example 1, are equilibrated with an acid or a buffer, adjusted to pH below 6.5. Suitable is a 50 mM ammonium acetate solution, pH 6, which is also the standard buffer for determination of the pore size distribution. Preferred is a 0.5 M to 2 M monobasic acid, e.g., 0.5 M to 2 M hydrochloric acid.

Subsequently the composite comprising the protonated swollen polymer is transferred into a solvent immiscible with water, stepwise as described above, preferably into toluene or n-butylether and aspirated, preferably followed by drying. The composite material, preferably comprising particles, is then transferred into a solution containing an excess of lipophilic anhydride reagent in toluene, preferably benzoic anhydride or methyl valeric anhydride.

Provided alkyl halogenides are used as derivatization reagents, the basic character of the polymer will be conserved, while the mesh becomes increasingly lipophilic, as the reaction proceeds. For this purpose alkyl bromides or aryl alkyl bromides are preferred. More preferred are alkyl bromides with four to 18 carbon atoms chain length.

In this particular embodiment the reagent is distributed between the composite pores and the external volume. The concentration of the functional groups of the immobilized polymer is usually between 0.5 M and 2 M. Hence, in preferred embodiments, also in combination of any of the above and below embodiments, the concentration of the derivatisation reagent is preferably 0.5 M, more preferred 1 M, most preferred 2 M, in order to achieve a high degree of derivatization.

After shaking for a sufficient time, preferably for 5 to 20 min, the reagent will be equally distributed between the pores and the external volume of the suspension. After aspiration, the composite is transferred into an aqueous solution of triethylamine, preferably between 0.5 M and 1 M. The dissolved strongly basic tertiary amine permeates the composite pores and converts protonated primary amino groups, protruding from the interior polymer surface, into their corresponding free basic form, which in turn readily reacts with the anhydride. At the same time the amino groups on the composite outer surface do not come into contact with the lipophilic anhydride, because they are exclusively wetted with water and thus remain vastly unchanged.

The above mentioned rules and principles regarding swelling, protonation, deprotonation and concentration of the compounds are recommended/mandatory for the related embodiments described below and above.

Embodiment According to Route 2)

Synthesis of a composite material which is lipophilic inside the pores, but anionic on the external surface.

The interior functional groups in the pore volume are derivatized according to the embodiment under route 1) above. The external functional groups are then derivatized according to the protocol for the embodiment under route 4) below.

Alternatively a poly(acrylate) or a poly(methacrylate) dissolved in water or preferably in an aqueous buffer solution, adjusted to a pH above 7, is filled into the pores of a support material. The suspension is aspirated and preferably dried. Subsequently the solvent is stepwise exchanged, beginning with ethanol, followed by acetone, toluene and eventually dichloromethane.

A solution of an activation reagent, preferably carbonyldiimidazole (CDI), more preferred an alkyl chloro carbonic ester is distributed in the pores, preferably by diffusion.

For this purpose the activation reagent is offered in a small volume of said solvent, preferably in excess concentration, more preferred with a 1.5 to 2 fold excess.

Alternatively, the support material containing the acidic polymer is dried, and the solution with the activation reagent is filled in the pores.

As soon as the activation of the carboxylic groups is completed, whereas the reaction time is preferably between 15 min and 30 min, followed by aspiration the polyacrylate is preferably cross-linked, using an amount of cross-linker equivalent to the targeted degree of cross-linking. The cross-linker is a diol, preferably a diamine.

In another embodiment, in combination with any of the above and below embodiments, the particles with the activated polyacrylate are dried under mild conditions, preferably at reduced pressure between 20° C. and 40° C., and the cross-linker solution is rapidly filled into the pores.

For the final derivatisation step it is also preferred to start from dried composite. This procedure enables the rapid distribution of the reagent all over the polymer.

Finally the remaining activated carboxyl groups are allowed to react with an aliphatic or aromatic nucleophilic compound, dissolved in an organic solvent, preferably with an alcohol, thiol or an amine, more preferred with benzylamine.

In a further embodiment, in combination with any of the above and below embodiments, the cross-linker and the derivatisation reagent are introduced simultaneously at the desired ratio as a mixture.

This procedure is applicable also with other polymers, cross-linkers, and derivatisation reagents comprising the above and below embodiments.

Embodiment According to Route 3)

Synthesis of a composite material which is lipophilic inside the pores, but inert on the external surface.

The interior functional groups in the pore volume are modified by derivatization according to the embodiment under route 1) above. The external functional groups are then derivatized according to the protocol for the embodiment under route 4) below, whereas the anhydride of dicarboxylic acids is replaced by acetic anhydride or a lactone.

Embodiment According to Route 4)

comprising selective reactions outside of the pores.

Synthesis of a composite material which is cationic inside the pores, but anionic on the external surface by selective derivatisation of the outer functional groups.

In one preferred embodiment, in combination with the above and below embodiments, the pores of a precursor composite material filled with cross-linked poly(vinylamine), e.g., according to example 5, are equilibrated with 50 mM ammonium acetate solution, pH 6, or preferably with a monobasic acid, thus protonating amino groups. When acid is used the particles are subsequently washed two times with water, in order to remove excess acid.

After careful aspiration of the suspending liquid, the particles, while still filled with the aqueous solvent, are mixed with an excess of a lactone or a an anhydride, preferably succinic anhydride solution of at least 100 mM and triethylamine (at least 100 mM) in an organic solvent, immiscible with water, preferably toluene, dichloromethane, or di-n-butylether and allowed to react for a short time, preferably between 5 min and 30 min.

The protonated amino groups of the polymer chains, protruding into the organic solution, are converted into free amino groups under these conditions and thus react with the anhydride, whereas a portion of the excess anhydride, regardless of being scarcely soluble in water, will become hydrolysed at the interface. The amino groups inside the pores, thoughalsopartially depronated, will nevertheless not become derivatized, or only to a minor extent, due to the vast absence of the poorly water soluble anhydride.

Embodiment According to Route 5)

Synthesis of a composite material which is cationic inside the pores, but inert on the external surface.

According to the above protocol of route 4), acetic anhydride or acetyl chloride are used within another preferred embodiment, in combination with the above and below embodiments, in order to obtain inert groups on the external surface not significantly interacting with any kind of biopolymer in the feed.

Embodiment According to Route 6)

Synthesis of a composite material which is cationic inside the pores, but lipophilic on the external surface.

According to the above protocol of route 4), a lipophilic anhydride, preferably benzoic or methyvaleric anhydride are used within another preferred embodiment, in combination with the above and below embodiments, in order to obtain a lipophilic external surface, not significantly interacting with any kind of solutes from organic solvents, but binding non-polar and medium-polar solutes from aqueous solvents.

Additional Chemical Modifications Inside the Pores.

Embodiment According to Route 7)

Synthesis of a composite material which is anionic inside the pores, but cationic on the external surface.

In one preferred embodiment, in combination with the above and below embodiments, a composite filled with swollen protonated cross-linked poly(vinylamine), at a pH below 6, is transferred from aqueous to non-aqueous conditions by solvent exchange from aqueous suspension to an organic solvent, preferably toluene or dichloromethane, followed by thorough aspiration, or preferably drying. The aspirated or dried composite is soaked in a solution containing an activated ligand, preferably a chloride or an anhydride of a multivalent carboxylic, sulphonic or phosphonic acid, preferably succinic or glutaric anhydride, in a non-water miscible solvent, preferably toluene, dibutylether, or dichloromethane, in order to distribute the dissolved reagent in the composite pores (as described above). Then the material is immediately aspirated again and washed with water, care being taken not to allow more than a very small portion of organic solvent from the interstitial particle volume to arrive at the top of the settled suspension, while water will not significantly flush the pores. Then the amino groups are converted from the cationic state into their free base condition, by shaking the particles in 1 M aqueous solution of preferably an organic base, e.g., triethylamine in water. While the triethylamine mediated generation of free amino groups inside the pores facilitates their rapid reaction with the water immiscible anhydride, the amino groups protruding from the outer particle surface into the aqueous phase are not noticeably derivatized, due to the respective lack of the reagent in this place.

After a sufficient reaction time the particles are aspirated and subsequently washed first with acetone, then ethanol, until the smell after toluene has disappeared. Eventually the material is washed with water and equilibrated in aqueous buffer solution for storage.

The preferred reaction time is 30 min up to two hours.

Embodiments of composites without polymer layers inside pores, according to the design criterion B 1.2 above.

An important class of composite materials of the present invention is comprising a bare adsorptive support material, harbouring pores of appropriate average size to realize a well-defined desired exclusion limit, externally coated with at least one polymer layer, preferably at least one inert layer n+i (i=1, 2, 3, k), whereas said external inert polymer is spatially excluded from the support pores, under the conditions of composite preparation.

However, most commercially available polymers, in particular those of technical grade, exhibit a broad molecular weight and thus radius of gyration (Rₐ) distribution. As a consequence, only polymer molecules small enough, will enter the support pores. In this way it is hardly possible to establish a clear division between polymers attached to the inner and the outer particle space. Accordingly the targeted design will not be preferably realized in this way. This is why it is advantageous to cut-off the low diameter polymer fraction. Polymer separation by gravimetric sedimentation or fractionation by size exclusion is not really feasible when dealing which large quantities.

One fast and reasonable operation is therefore disclosed within the present invention: In one preferred embodiment, also in combination with each of the above or below embodiments, the undesired low molecular mass polymer fraction is removed from the solution by contacting said solution with an adsorbent, exhibiting an upper pore size limit establishing the targeted exclusion limit. In this case the smaller molecular polymer coils become readily adsorbed inside the pores of said adsorbent, while only a negligible portion of larger polymer molecules bind to the outer surface, because the latter represents only a small portion of the total available surface of the material. Subsequently the solution is removed by filtration, sedimentation, or centrifugation, leaving the solid material ready-to-use for the subsequent coating of the outer surface.

The preferred adsorbent for the removal of said low diameter polymer molecules later serves also as a support material for the attachment of the inert layer.

Provided that the lower fraction cut-off is not complete, the adsorption procedure may be repeated.

The preferred adsorbents for this kind of polymer fractionation are alumina, titanium dioxide, and more preferred silica gels with a nominal pore diameter between 2 nm and 100 nm, preferably between 5 nm and 50 nm, more preferred between 10 nm and 30 nm, also in order to take advantage of their high binding capacity for any polar compounds in a separation process. The proper porosity as well as the necessary amount of such adsorbent for the removal of the low molecular weight fraction in an intended particular separation process are figured out according to data from inverse size exclusion chromatography (iSEC) of the respective polymer solution, gathered in advance.

Such polymer loaded silica can serve for many adsorption processes with lower selectivity requirements, e.g., the depletion of hazardous substances from diluted waste solutions, or, more generally, in a variety of waste-water treatments.

Subsequently the polymer of choice is immobilized to the external surface of the desired support material.

In a preferred embodiment, in combination with any of the above and below embodiments, the support material is filled with water and aspirated from the supernatant, before the filter cake is suspended in an aqueous solution of the polymer. After aspiration, the polymer coated particles are suspended with twice of its volume of a solution of the cross-linker dissolved in an organic, not water miscible solvent, preferably aspirated again, heated and kept at 50° C. to 90° C. for 10 min to 120 min. This reaction may also be carried out in suspension, prepared in excess solution of the cross-linker, commonly resulting in a comparatively higher degree of cross-linking.

Alternatively, in another embodiment, in combination with any of the above and below embodiments, the cross-linker is dissolved or suspended in the polymer solution, the resultant reaction mixture is contacted with the support material, aspirated and the coated support material is heated.

In another preferred embodiment, in combination with any of the above and below embodiments, the pores of the support material are filled with a water immiscible solvent and then aspirated. Subsequently the aqueous solution with the shrunken polymer, preferably a polyamine or a polyalcohol, together with a cross-linker, preferably a bis epoxide, are mixed with the support material and aspirated after 5 min to 10 min. A layer comprising the polymer plus cross linker remains adsorbed to the exterior surface of the support material. Cross-linking is then achieved by heating the coated material at 50° C. to 120° C. for a sufficient time, as described for the epoxycross-linkers above. Preferably the organic solvent is evaporated from the pores before the cross-linking reaction is started.

In a further embodiment, in combination with any of the above and below embodiments, the polymer is adsorbed to the exterior surface of the support particles which are filled with the organic solvent, but without adding the cross-linker at the beginning. Then the organic solvent is preferably evaporated and the dry particles are briefly dipped in an organic solution of a cross-linker, preferably a bis-epoxide. Hence the polymer layer becomes loaded with an appropriate amount of the cross-linking agent. After aspiration the particles are heated for 10 min to 2 hours at a temperature between 110° C. and 60° C.

The support used for the embodiments with materials, which are only coated on the exterior surface, is preferably comprising a porous material with a nominal pore diameter of 4 nm to 100 nm, preferably of 10-50 nm, more preferred of 15-30 nm.

Preferred are inorganic and organic monolithic or particulate porous materials, more preferred are particles, most preferred are silica gel, alumina, titanium and zirconium oxides or cellulose, dextrane gels, polyacrylic and polyester materials, all of them harbouring pores within said range of pore size.

In one preferred embodiment, in combination with any above and below embodiments, a functional polymer, preferably a polyamine, more preferred poly(vinylalcohol) or a co-polymer thereof, exhibiting a molecular mass of at least 100,000 Da/a Rₕ value of at least 9 nm in the solvent used for the synthesis, is attached to a support material, preferably after the low molecular weight fraction was removed according to the procedure described above.

In another preferred embodiment, in combination with any above and below embodiments, a silica gel with pores between 10 nm and 100 nm is contacted with a solution of poly(vinylalcohol) with a molecular mass above 100,000 Da, preferably 200,000 Da, more preferred above 500,000 Da for a sufficient time. After removing the polymer solution preferably by aspiration, the particles are washed with dichloromethane, whereas the water remains inside the pores. They are then suspended in a solution of an at least bivalent epoxide, preferably of hexane diol diglycidyl ether, in a solvent not miscible with water, preferably toluene or dichloromethane, and heated at a temperature between 50° C. and 130° C. during a time between 10 minutes and 24 hours, until the remaining external polymer layer is cross-linked. Finally the composite material is washed with ethanol, water, and the buffer appropriate for the particular application. The degree of cross-linking is dependent on the cross-linker concentration in the chosen solvent. The amount of reacted cross-linker is preferably determined by the time dependent concentration measurement of the cross-linker solution using the established gas chromatographic methods.

In order to adjust the degree of cross-linking in a system with support material, polymer and cross-linker for a first time, the amount of immobilized polymer is measured using thermo-gravimetry, and four test batches are synthesized with different cross-linker concentration. Finally this concentration may be adjusted according to the target degree of cross-linking.

Reactions with Activated Polymers or Activated Reagents.

In a preferred embodiment, in combination with the above and below embodiments, poly(acrylate) with high molecular weight is attached to the external surface of the composite with a cross-linked polyamine as a first layer. The immobilisation of poly(acrylates) readily takes place in aqueous solutions resulting in the formation of a thin polymer layer adsorbed by ionic forces. By reaction with remaining epoxide residues of the cross-linker the carboxyl groups of the polyacrylate will form an ester of poor stability, however.

Alternatively the usual activation methods known from peptide chemistry may be applied for the carboxyl-amine coupling, e.g., with water soluble carbodiimide or with a chloro carbonic alkylester, after exchanging the solvent to dichloromethane or toluene, forming mixed anhydrides.

The use of poly(maleic) anhydride as a precursor of an inert external n+1 layer is preferred, due to the rapid formation of amide bonds with the protruding amino group exposing polymer chains, whereas a preferably tertiary organic amine in the reaction solution both converts the ammonium ions to amino ligands and neutralizes the second maleic acid residue. Excess anhydride groups are finally quenched with, e.g., aminoethanol. Dependent on the solubility of the particular polymer or co-polymer this reaction will require a water immiscible organic solvent as extraparticle medium, while the pore volume remains filled with water. Alternatively both the internal and the external volume may be filled with an aprotic organic solvent, preferably dimethylformamide (DMF).

In a preferred embodiment, in combination with the above and below embodiments, the composite design is anionic inside the pores, preferably comprising a hydrolysed maleic anhydride polymer, and cationic on the particle surface, preferably after the reaction with a polyamine of adequately large molecular size to remain sterically excluded from the pores.

Basically it is possible to derivatize the first polymer inside the pores and then attach the second polymer after an additional activation step. As small molecules, like bi-valent activators of the carbonyldiimidazole type (CDI) or epichlorohydrine (ECH), will always have access to the pores, the original functional groups inside the pores become also activated or even cross-linked to a certain extent.

In order to avoid undesired cross-linking, excess activation reagent is used (1.5 equivalents in excess, at least). Although principally feasible, any stoichiometric activation is significantly more difficult.

Provided that the initially present functional groups are not essential for the desired interaction with solutes, their chemical modification, i.e., conversion into different functional groups may be considered acceptable. Finally, after introduction of the desired ligand, excess active sites should be de-activated. If the activation of poly(vinylamine) was, e.g., performed with CDI, aminoethanol will eventually generate hydroxy groups, or de-activation using (diethylamino)ethylamine will even keep the basic character of the initial polymer.

This activation may additionally serve for the later introduction of other desired functional groups/ligands.

In a preferred embodiment, in combination with the above and below embodiments, it is also possible to derivatize one or both polymers in advance before the immobilisation. At higher degrees of derivatisation the subsequent cross-linking and the binding of both polymers is more difficult, however.

Basically the derivatisation of a polymer mesh is applicable in any of the above embodiments, including utilization of alternate support materials, e.g., monoliths, various kinds of tissue or any kind of filter materials.

Moreover, also even or flat-shaped devices selected from a variety of materials are applicable in the described selective derivatisation procedures, provided they are compatible for moistening with the respectively immiscible solvents or solutions.

Embodiments Relating to the use of the Adsorbents of the Present Invention

In one preferred embodiment, in combination with the above and below embodiments, any of the composites mentioned and any of the composites which can be made relating to the abovementioned design principles and methods may be used for the purification of a target compound from a feedstock.

Within further preferred embodiments, in combination with any of the above and below embodiments, at least two composites are mixed for the purification of a target compound, preferably protein from a feedstock. The ratio is arbitrary and can be estimated and adjusted according to the particular purification task. Preferred ratios are in the range between 10:90 to 90:10, when two composites are mixed.

In a preferred embodiment, in combination with any of the above and below embodiments, a selection of at least two composite materials are thoroughly mixed and used in a batch separation process.

The ratio of composite materials in the mixture is selected following a thorough consideration of the impurity profile of the respective sample or process feed. In order to achieve a complete depletion of impurities the depletion rates with the individual composite materials are to be assessed first. In addition, isoelectric focusing of collected and further concentrated impurities, as isolated, e.g., from previous runs, allows to pre-select suitable composite adsorbents and suitable combinations thereof.

The various composites and composite mixtures can be applied in a column, comprising any kind of chromatographic process, preferably gradient elution or isocratic elution liquid chromatography, but also in expanded bed and fluidized bed techniques.

In a preferred embodiment, in combination with any of the above and below embodiments, at least two composite materials are thoroughly mixed and the resulting blend packed into a chromatography column.

Preferably a batch separation process is applied avoiding convective flow operations. More preferred is a two step batch process, most preferred a one step batch process.

With respect to the abovementioned various separation processes a combination or system of at least two composite materials is applied.

Moreover, a system comprising at least one composite material and at least one liquid phase is applied.

Other General Synthesis Principles and Methods tor the Composite Adsorbents and Related Embodiments.

It is the objective of the present application to provide methods for the preparation of a composite material.

The objective is achieved by a process, comprising:

Filling at least the pore volume of a porous support material with a solution of at least one functional polymer or co-polymer and optionally at least one cross-linking agent (reaction mixture), and in situ immobilizing said functional polymer by cross-linking, co-valent attachment, or precipitation, whereas the support material is particulate, pellicular or monolithic.

As a support material also a composite adsorbent may serve of another preparation or of commercial origin.

The following principles and the relating embodiments of preparation are both applicable for the immobilisation of the first polymer 1 and of further adsorptive polymers 2, 3 , n, as well as for the immobilisation of non-adsorbing polymers n+1, n+2 . . . n+i. Thus the following are preferred embodiments of the preparation of said composite materials according to the present invention:

If a polymer or copolymer is functionalized, it exhibits at least one group per molecule capable for cross-linking, or co-valent binding to the support surface, or adsorption on this surface.

In combination with any of the above or below embodiments, any cross-linker known from prior art is applicable for the immobilization of a polymer according to the present invention.

In combination with any of the above or below embodiments, the cross-linker is preferably a bis-oxirane or a bis-aldehyde such as succinic or glutaric dialdehyde, as long as the polymer is harboring amino groups. If a bis-aldehyde is used as the cross linker, a subsequent reduction step is advantageous for stabilisation purposes. Cross-linkers with more than two reactive groups are also applicable.

Preferably the cross-linker should represent the chemically activated reagent in the formation of the polymeric mesh.

Alternatively, the polymer may be introduced as the chemically activated partner, using the reagents and procedures as known from the prior art, in particular from peptide synthesis.

The polymer may also a priori be reactive. In this case functional groups of the polymer may be generated during the cross-linking process itself or subsequently, applying reactive or activated polymers, e.g., anhydrides from poly(maleic acid), or poly-oxiranes.

Both, cross-linker or polymer may also be activated using the prior art carbodiimide reagents, preferably the water soluble carbodiimides, in order to allow the whole reaction to take place under aqueous or non-aqueous conditions.

Any solvent may be used for the synthesis, which does either not react or only slowly reacts with the cross-linker and/or the cross-linkable polymer under the conditions of preparation, and which preferably dissolves said reactants to at least 1% (w/v) solution. Slowly in this context means that at the selected temperature no visible gelling occurs before at least 30 minutes, using only the polymer cross-linker solution as demonstrated with Comparative Example 1.

It is advantageous for the synthesis process and the subsequent wash and equilibration to use only aqueous media, applying preferably cross-linkers soluble in water or miscible with the aqueous reaction solution.

In a preferred embodiment, in combination with any of the below embodiments, the cross-linking reaction is not started already during the pore filling, but subsequently, preferably at elevated temperature or with a pH shift. The cross-linking with epoxide cross-linkers or epoxy-activated polymers is thus started at temperatures preferably above 50° C., while at room temperature no visible gelation occurred after 30 minutes, even not after two hours.

Within a further preferred embodiment, in combination with any of the below embodiments, the cross-linking of amino containing polymers with reactive cross-linkers like carbonyl diimidazole is suppressed at pH values below 7, preferably below 6, and will be started after adjusting the pH above preferably 7, more preferably 8, because the reaction velocity of the protonated amino groups is very low.

Within another embodiment, in combination with any of the below embodiments, the cross-linker is applied first into the support material pores, optionally the resin is at least partially dried, and finally the polymer solution is introduced and cross-linked.

In a more preferred embodiment, in combination with any of the above or below embodiments, the cross-linker is applied in water or in an aqueous solution together with the cross-linkable polymer. Although even cross-linker quantities below 2% (v/v), preferably using the abovementioned bis-epoxides, most preferred hexanediol diglycidylether are not completely soluble in water, the emulsion formed surprisingly distributes inside the support material pores, thus generating a stabile cross-linked polymeric mesh.

In a preferred embodiment, in combination with any of the below embodiments, the object of the present invention is reached by the reaction of at least one shrunk crosslinkable polymer with at least one cross-linker, thus forming at least one polymeric layer, comprised in a mesh, which is selectively swollen or shrunk in certain solvents or buffers.

This is the preferred way how to attach the first polymeric layer.

Alternatively, in a different embodiment, in combination with any of the above or below embodiments, the first polymer may be covalently attached to the surface of the support material, and optionally cross-linked in addition.

In further preferred embodiments, in combination with any of the below embodiments, any subsequently attached polymer is either connected by covalent or non-covalent binding to the preliminary polymeric layer, or it may be independently cross-linked.

In a preferred embodiment, in combination with any of the below embodiments, the pores are filled with a reaction solution comprising the functional polymer and optionally the cross-linker, and reacted in a one step process without preliminary or intermediate drying.

In another preferred embodiment, in combination with any of the below embodiments, the present invention is related to pore filling steps with a reaction solution prepared with a non-swelling solvent, solvent mixture or buffer.

For the purpose of reaction including preliminary pore filling, the cross-linkable polymer or co-polymer is preferably dissolved in a solvent or buffer which will shrink the polymer. Thus the molecular volume of the individual polymer coils or bodies will be minimized, allowing introducing a maximal amount of polymer into the narrow pores.

In the case of polyacrylates, or other acidic polymers swelling is suppressed within the acidic pH range, generating a non-dissociated configuration. In the case of amino containing polymers a basic pH generates this desired molecular shrinking. Neutral polymers, like polyvinyl alcohol, are preferably dissolved in aqueous mixtures close to the theta point, e.g., with water-propanol mixture.

In a preferred embodiment, in combination with any of the below embodiments, the support material is filled with the reaction solution applying the spontaneous soaking of the liquid into the pores. Any other method of pore filling known from the prior art is also applicable.

Using soaking techniques, it is difficult to fill exactly the entire pore volume of porous particle support materials, however. As also the pore volume determination (compare Methods) will always imply a certain error, it becomes even more difficult to accurately determine the necessary volume of reaction liquid to be applied. Therefore one can hardly avoid that a significant fraction of the particles will become slightly overloaded with liquid on the outer surface, e.g. simply because of the surface tension of the liquid. As a consequence, pore portions of other particles inevitably will not be completely filled, when only a volume of the reaction mixture is added, which is equal to the pore volume determined.

As it is basically impossible during the manufacturing process to contact the particles all at once with the liquid, this problem of inhomogeneous particle filling will even become more severe, in particular while treating large quantities.

With respect to the dedicated applications, it is the absolute request to completely cover the accessible surface of a support material with the cross-linked polymer.

Thus the abovementioned kind and extent of inaccuracy is not negligible. Support surface fractions which are not covered with polymer will have a negative impact on the selectivity and mainly recovery during a separation process. There may be a stronger adsorption of the target compound on these spots, in particular with protein targets compounds on polar support materials like silica or other polar media.

Said problems can be avoided applying a sufficient excess of reaction mixture volumes, enabling the complete wetting and polymer coverage of the entire support surface. In this case, however, it will be necessary to prevent any cross-linking reactions outside of the particle volume. Moreover, also no rapid reaction of the polymer and the cross-linker is acceptable during a sufficient pot life time after the preparation of the reaction mix.

Surprisingly it has been found that even when the interstitial volume between particles partially or completely contains the reaction solution, not any particles are fused together.

Without being restricted to any explanations, the cross-linker is probably adsorbed by the porous support material in this case (Example 1). Accordingly the composite preparation is not negatively affected if a certain excess of polymer cross-linker solution is applied. These unexpected findings allow a simplification of the manufacturing process according to the present invention, especially while producing large quantities of composite material, because the reaction preferably will be carried out with the sedimented support material without stirring, shaking, or other movement.

Therefore, in combination with any of the above or below embodiments, at least the pore volume of a support material is filled with the reaction solution, preferably an excess solution related to the pore volume, more preferred the sedimentation volume, and most preferred a slight excess of the sedimentation volume are added.

Therefore, in combination with any of the above or below embodiments, a solution of the functional polymer, preferably poly(vinylamine) or poly(vinylformamide-co-vinylamine), or poly(vinylalcohol), or co-polymers thereof, together with a cross-linker, preferably a bis-epoxide, more preferably ethyleneglycol-, propyleneglycol-, butanediol-,or hexanedioldiglycidylether, is offered in amounts of at least the pore volume, preferably of the sedimentation volume, and most preferred between 110% and 120% of the overall sedimentation volume, whereas the pores of the support material became completely filled. Unexpectedly at the end of the reaction neither polymer gel was formed outside of the pores nor did the particles glue together.

In a preferred embodiment, also in combination with any of the above or below embodiments, an excess of the cross-linker containing solution of a functional polymer, preferably between 110% and 120% of the support material sedimentation volume is added to the support material, so that the interstitial volume between the particles is completely filled with liquid, and a thin liquid film of reaction solution covers the top of the sedimented solids.

Thus the present invention is also providing a process for the preparation of a composite material comprising:

Filling at least the pore volume of a porous support material or of a dried composite adsorbent with a solution of at least one cross-linkable polymer or co-polymer and at least one cross-linking agent (reaction mixture), and in situ immobilizing said cross-linkable polymer by cross-linking, wherein the support material or the dried composite adsorbent is particulate, pellicular or monolithic.

By introducing polymer solutions, with or without cross-linker, to a dried precursor composite adsorbent the next polymeric layer is synthesized.

In a preferred embodiment, in combination with any of the below embodiments, the cross-linkable polymer is poly(maleic anhydride) and its co-polymers, poly(methacrylic acid), poly(acrylic acid), poly(vinylalcohol) and related co-polymers, poly(vinylformamide-co-vinylamine) or poly(vinylamine), or a mixture thereof.

One-step and in situ means, that all reactants are mixed, reacted, and the composite is washed within one working operation, in order to obtain the desired product. Mainly the immobilization via cross-linking is achieved at once with or after the application of the complete reaction mixture.

In a preferred embodiment, in combination with any of the below embodiments, the reaction mixture is containing salt, buffer and/or other compounds, which are not incorporated in the composite products. Accordingly technical grade polymers and cross-linkers may be applied, even contaminated with various side products.

Provided that the support material is an assembly of monolithic items, e.g. a stack, said process is preferably comprising:

Filling at least the pore volume and the interstitial volume between the layers with said reaction mixture.

In a preferred embodiment, in combination with any of the below embodiments, the polymer immobilisation is achieved according to the above procedure, whereas the reaction mixture is containing salt, buffer and/or other compounds, which are not incorporated in the composite products. This will allow to use polymers and cross-linkers of technical grade or related raw materials.

The amount of polymer introduced into the support material and immobilized is preferably controlled by the polymer concentration in the respective reaction solution. The degree of support pore filling and the mesh size distribution under application conditions, in contrast, is controlled by the solvent-dependent swelling of the polymer and its total immobilized amount. Both parameters taken together, the overall amount of polymer immobilized and the degree of swelling allow adjusting the percentage of the overall pore volume which is filled with the polymer.

For synthesis purposes the degree of filling is exactly determined and standardized by weighing the wet and dry materials before and after introduction of the polymer-cross-linker solution.

In a further preferred embodiment, in combination with any of the above or below embodiments, the degree of support pore filling and the mesh pore size distribution under application conditions is achieved and determined by introduction and immobilization of different polymer amounts and by the subsequent measurement of the pore size distribution. The amount of polymer to be immobilized is preferably adjusted by the polymer concentration in the reaction solution. Hence, the maximal possible polymer amount, which can be immobilized, or any appropriate amount, is easily elucidated for said purpose.

In combination with any of the above or below embodiments, the present invention is providing methods for the synthesis and the use of a polymeric mesh exhibiting an upper, but variable pore size Rₕᵢ, when equilibrated with an appropriate solvent, thus capable of retaining a significant amount of compounds with a hydrodynamic radius below this exclusion limit Rₕᵢ (nm) inside the pore volume, preferably 50%, more preferred 80%, most preferred >90% of the initial content, whereas the pores of the polymeric mesh are not accessible for the at least one target compound with a hydrodynamic radius of or above Rₕᵢ and thus allows to recover said target compound in the solution, preferably in the purified feed.

Said object of combining sorption, partitioning, and size exclusion is preferably achieved by the use of a composite material or a mixture of composite materials comprising: At least one porous support material having an average pore size between 5 nm and 5 mm, wherein the overall pore volume of the at least one porous support material is filled with at least one polymer, which is cross-linked and thus forming a mesh, which is excluding standard molecules of a hydrodynamic radius Rₕᵢ (nm) and thus provides an exclusion limit for synthetic and natural macromolecules with a hydrodynamic radius of Rₕᵢ or above Rₕᵢ (nm), when equilibrated with an appropriate solvent.

Provided that the target compound is an antibody, said exclusion effect is achieved if this mesh is inaccessible for molecules exhibiting a hydrodynamic radius Rₕ₁ above 5 nm, preferably above 4 nm.

The related reaction time for each polymer or layer immobilisation step is preferably between 10 min and 100 hours, more preferably between 30 min and 48 hours and most preferred between 10 min and 24 hours.

The range of temperature for the synthesis of the composite materials is preferably between 20° C. and 180° C., more preferably between 40° C. and 150° C., and most preferably between 60° C. and 110° C.

Preferred Embodiments Relating to the use of the Composite Materials.

In combination with any of the above or below embodiments, the present invention is providing materials and methods for the use of the composite adsorbents, achieving a simultaneous removal of several structurally different classes of substances from a solution, preferably a feedstock, whereas at least one target compound remains substantially unbound and is recovered at a high yield.

This target compound yield is preferably 80%, more preferably 90%, and most preferred above 95%.

The separation methods of the present application for recovering a target compound from a feedstock are preferably comprising the following steps (i) to (iv):
(i) providing at least one porous composite material comprising at least one polymeric layer, said polymeric layer is comprising at least one immobilized polymer, and the porous composite material being characterized by a pore size exclusion limit Rₕᵢ which can be set variably;
(ii) optionally adapting the variable pore size exclusion limit Rₕᵢ of the porous composite material to the hydrodynamic radii of the target compound Rₕᵢ and the at least one impurity Rₕ₂ such that R_{h2<}Rₕᵢ and Rₕ₁>Rₕᵢ;
(iii) contacting the composite material with the feedstock for a time sufficient to allow retaining the impurity compound in the polymeric mesh and excluding the target protein from the polymeric mesh;
(iv) separating the composite material containing the retained impurity compound from the feedstock containing the excluded target protein in order to obtain a purified feedstock;

Optionally, the target protein is isolated from the purified feedstock.

The composite adsorbent is used either in a chromatographic process or within a one-step batch separation.

The present application is also related to a method for recovering a target compound from a feedstock, said feedstock being in the form of a solution or suspension, and being preferably a fermentation broth, and comprises at least one target molecule, preferably a protein, more preferably an antibody and at least one impurity compound, preferably selected from host cell proteins (HCP), DNA, RNA or other nucleic acid, or a combination of two or more thereof, and optionally comprising albumins, endotoxins detergents and microorganisms, or fragments thereof, or a combination of two or more thereof, said method comprising the steps of:
i) contacting said feedstock with a composite adsorbents according to any of the preceeding claims for a sufficient period of time, wherein at least one impurity compound is retained;
ii) subsequently, separating the composite adsorbent from the purified feedstock containing at least one target compound;
iii) optionally, isolating the target compound from the feedstock.

Moreover is the present application also related to the following methods and procedures:

A method for recovering a target compound from a feedstock according to the proceeding method, wherein the target compound is a target protein being characterized by a hydrodynamic radius Rₕi and the impurity compound being characterized by a hydrodynamic radius Rₕ₂, wherein Rₕ₁>Rₕ₂, the method comprising the following steps (i) to (iv) and optionally step (v):
(i) providing a composite adsorbent, exhibiting a pore size exclusion limit Rₕᵢ which can be set variably;
(ii) adapting the variable pore size exclusion limit Rₕᵢ of the composite adsorbent to the hydrodynamic radii Rₕ₁ and Rₕ₂ such that R_{h2<}Rₕᵢ and Rₕ₁>Rₕᵢ;
(iii) contacting the composite adsorbent with the feedstock for a time sufficient to allow retaining the impurity compound in the composite adsorbent and excluding the target protein from the composite adsorbent;
(iv) separating the composite adsorbent containing the retained impurity compound from the feedstock containing the excluded target protein in order to obtain a purified feedstock;
(v) optionally, isolating the target protein from the purified feedstock.

A method for recovering a target compound from a feedstock according to the proceeding methods, wherein the target compound is a target protein being characterized by a hydrodynamic radius Rₕ₁ and the impurity compound being characterized by a hydrodynamic radius Rₕ₂, wherein Rₕ₁>Rₕ₂, the method comprising the following steps (i) and (iii) to (iv) and optionally step (v):
(i) providing the composite adsorbent comprising at least one polymer, the composite adsorbent being characterized by a pore size exclusion limit Rₕᵢ such that R_{h2<}Rₕᵢ and Rₕ₁>Rₕᵢ;
(iii) contacting the composite adsorbent with the feedstock for a time sufficient to allow retaining the impurity compound in the composite adsorbent and excluding the target protein from the composite adsorbent;
(iv) separating the composite adsorbent containing the retained impurity compound from the feedstock containing the excluded target protein in order to obtain a purified feedstock;
(v) optionally, isolating the target protein from the purified feedstock.

A method, wherein said above setting in step (i) or said adapting in step (ii) or said setting in step (i) and said adapting in step (ii) is performed by one or more of the following: varying the structure of the polymer, selecting the cross-linker used to generate a crosslinked polymer, selecting the degree of cross-linkage of the polymer, controlling the degree of swelling of the polymer by varying the solvent for the preparation and the use of the polymer, particularly varying the pH of the solvent and thus the degree of protonation of the polymer, and controlling the concentration and the immobilized amount of the polymer within said adsorbent composite.

A method related to any one of the preceeding methods, wherein said crosslinked polymer contains positively charged amino groups.

A method of any one of the preceeding methods, wherein the at least one impurity compound retained by the composite adsorbent exhibits a hydrodynamic radius Rₕ₁ that is lower than the hydrodynamic radius of the target compound, preferably protein remaining in the purified feedstock, preferably wherein the at least one impurity compound retained by the composite adsorbent exhibits a hydrodynamic radius Rₕ₁ below 4 nm, wherein preferably said impurity compound is a host cell protein, and wherein the at least one target compound, preferably protein remaining in the purified feedstock exhibits a hydrodynamic radius Rₕ₁ of 4 nm or greater than 4 nm.

Another method relating to of any one of the preceding methods, comprising: equilibrating the composite adsorbent obtained in step (ii) prior to the contacting in step (iii) to a pH below 8; or equilibrating the composite adsorbent provided in step (i) prior to the contacting in step (iii) to a pH below 8.

A further method of any one of the preceding methods, wherein step (iii) comprises: depleting neutral or positively charged compounds with a pI (isoelectric point) of 7 or above 7 by the equilibrated composite adsorbent.

Method of any one of the preceding methods, wherein the variable pore size exclusion limit Rₕᵢ of the composite adsorbent provided in step (i) and/or adapted in step (ii) is set or adapted to a range of from 1 to 20 nm, preferably is set or adapted to a range of from 3 to 10 nm.

A method for the use of a composite adsorbent according to any one of the preceding methods for recovering a target protein from a feedstock, said feedstock being in the form of a solution or suspension, and being preferably a fermentation broth, and comprises at least one target molecule, preferably a protein, more preferably an antibody and at least one impurity compound.

Chromatographic processes are comprising column, fluidized bed, expanded bed operations, or related techniques as known to a skilled person. Batch separation means that the feed is contacted with the composite adsorbent, preferably mixed and suspended, whereas the purified solution is removed after sedimentation or centrifugation.

In a preferred batch embodiment, in combination with any of the above or below embodiments, a certain volume of the particular feedstock (e.g., from a fermentation process before or after removal of the solid materials like a cell culture or its supernatant) is contacted with a sufficient amount of polymer containing composite material in suspension.

In a preferred embodiment, in combination with any of the above or below embodiments, the ratio of feedstock to composite material is in a range between 5 and 100 litre per kg, and the preferred contact time is 5 to 60 min.

The polymers of the composite materials of the present application are preferably swollen in an appropriate solvent or buffer before the use and subsequently dried. This dry state is also the favourable condition for storage.

For the swelling and equilibration purpose a composite adsorbent comprising chargeable ligands like amine or carboxyl is preferably neutralized with strong bases or acids. Aminopolymer containing composites are treated with preferably a 1.5 molar to 2 molar excess of preferably monobasic acids like formic, acetic, sulfamic, hydrochloric, or perchloric acid, preferably on a frit, digesting the resin bed with three volumes of the liquid. After aspiration this wash is repeated five times, followed by rinsing with water until the eluate is between pH 5 and 8.

The composite material is now ready for use or drying.

In a preferred embodiment, in combination with any of the above or below embodiments, hydrochloric acid is used for the conversion of basic groups, because the hydrochloride containing polymeric layer exhibits excellent adsorption capabilities for various impurities. Appropriate concentrations of the hydrochloric acid are between 0.2 M and 1 M.

Ammonium, alkyl ammonium, sodium, and potassium are preferred cations after the neutralization of acidic ligands in the composite adsorbent with the respective bases applying concentrations between 0.1 M and 0.5 M.

A composite adsorbent comprising zwitterionic ligands is preferably treated at pH values between 4 and 9 with a 500 mM buffer followed by the 50 mM buffer with the same composition. Preferred are sodium formate, sodium acetate, ammonium acetate, ammonium formate and ammonium chloride.

After aspiration of the liquid these composite adsorbents may be directly contacted with the feed solution. With respect to a batch process they are preferably dried and wetted only with one pore volume water before contacting with the feed.

For the purpose of column packing the dry stored resin is suspended in three to five bed volumes of water and packed in a column according to one of the usual procedures. When the depletion of the impurities is complete, the supernatant with the purified target compound is removed, preferably using centrifugation or decanting procedures. In chromatography, the column is loaded with the appropriate volume of feed avoiding the breakthrough of impurities. The flow-through fraction containing the target product is collected, and the product in the interstitial packing volume is optionally displaced with water or a very weak buffer below 10 mM.

Finally the composite materials are preferably disposed.

The above and the following further objects of the present invention are also achieved according to the bio-separation embodiments as outlined below.

The general task in bio-separation is the depletion of the substances as listed under a)-g) below. When a fermentation broth (before or after filtration) or a cell culture supernatant (CCS) is used as a feedstock containing the target compound, the target compound is a recombinant protein, preferably an antibody, and the feedstock comprises the following classes of compounds as impurities:
a) DNA, RNA, other nucleic acids, proteins, and organic substances with a molecular mass of at least 100,000 Dalton;
b) host cell proteins (HCP) inclusive proteases with a molecular mass below 100,000 Dalton
c) albumin (BSA, HSA, ovalbumin);
d) other proteins present in cell culture media as well as substances of various molecular masses which stem from nutrients or cell metabolism; 20
e) endotoxins;
f) detergents; and
g) germs and microorganisms such as viruses, or fragments thereof.

The following are preferred embodiments of the method for separating a target compound according to the present invention:

The separation method of the present invention preferably relates to a feedstock, e.g. a fermentation broth, representing either a filtrated solution or a raw suspension, still containing e.g. cells and cell debris.

In a preferred embodiment, in combination with any of the above or below embodiments, the target compound is one of the substances defined above.

In a preferred embodiment, in combination with any of the below embodiments, the undesired compounds are selected from DNA, RNA, albumins, host cell proteins (HCP), endotoxins, detergents, bacteria and viruses. Also fragments of said undesired compounds, like coating proteins, S-layers, cell fragments or debris are within the scope of this embodiment.

In a preferred embodiment, in combination with any of the above or below embodiments, the target compound is an antibody and only the impurities a), b) and c) listed above are depleted from the solution. In a further preferred embodiment in combination with any of the above or below embodiments, the target compound is an antibody and only the impurities a) and b), as listed above are depleted from the solution.

In a further preferred embodiment in combination with any of the above or below embodiments, the target compound is an antibody and only DNA and host cell proteins as impurities (undesired compounds) are depleted from the solution.

Preferably, the contaminants or impurities are depleted from a feedstock (e.g. biological fluid, supernatant of a fermentation process, or the fermentation broth before filtration) at a degree of >90%, >95%, >99% of their respective total amounts in the feedstock with concomitant binding of no more than 10%, preferably 5%, more preferably 1% of the total amount of target substances.

Accordingly, the present invention is related to a purification process comprising the steps i), ii), iii) and (iv), characterized in that the impurities are depleted to at least 90%, whereas the target protein is recovered to at least 90%.

In a preferred embodiment, in combination with any of the above or below embodiments, the host cell proteins are depleted to an amount of at least 90%, preferably to at least 95%, more preferred to at least 99%.

Accordingly, the present invention is related to a purification process wherein the host cell proteins are depleted from the feed to at least 90% of their initial concentration.

Pore volume in the context of the present invention means the integral or sum of the entire particular pore volume fractions, each of which fractions is defined by a lower and an upper pore size.

Also in combination with any of the above or below embodiments, the present invention is providing the synthesis and use of composite materials exhibiting a defined pore size distribution, capable of retaining a significant amount of compounds with a hydrodynamic radius Rₕ₂ below 4 nm within their mesh pore volume, preferably 50%, more preferred 80%, most preferred >90% of the initial content, whereas this fraction of the pore volume is inaccessible for target compounds with at or above 4 nm, like antibodies, and whereas another portion of undesired products with higher molecular weight is bound to the external surface.

The above-mentioned another undesired products are preferably microorganisms like bacteria and viruses, nucleic acids and/or host cell proteins with a molecular weight above 100,000 Da.

The above objects of protein purification are preferably achieved by the use of a composite material or a mixture of composite materials comprising:

at least one porous support material having an average pore size of 20 nm to 5 mm, wherein, under the conditions of application at a pH between 3 and 11, preferably between 5 and 9, the overall pore volume of each porous support material is filled with at least one cross-linked functional polymer, said composite at least one material is characterized by a pore size distribution, wherein molecules with a hydrodynamic radius Rₕᵢ of 4 nm and above, in particular the calibrated pullulane standard with a molecular weight of 21.7 kDa and Rₕ=3.98 nm, are excluded from at least 90% of the pore volume.

In combination with any of the above or below embodiments, the present invention provides the use of a composite adsorbent, comprising an adsorbing polymer, preferably poly(vinylamine) or poly(vinylformamide-co-vinylamine), a non-adsorbing polymer, preferably poly(vinylalcohol), and a cross-linker, characterized in that pullulane standards exhibiting a hydrodynamic radius above Rₕᵢ=3.98 nm are substantially excluded from the pore volume, thus defining the upper pore diameter in the respective solvent, whereas the mesh volume is accessible to a pullulane standard 6.2 kD with a hydrodynamic radius Rₕ₂=2.13 nm. Substantially excluded means that at least 90% of the pore volume is not accessible.

The pore accessibility and the exclusion limit are determined by iSEC using pullulane standards in 20 mM ammonium acetate buffer preferably at a pH between 6 and 9. (see Methods).

Combinations with other Adsorbents and Separation Methods:

In order to fulfil the stringent quality requirements in place for APIs (active pharmaceutical ingredients), the target compound purified according to the new technical lore may require one or two additional purification steps. This may be the case if depletion below the detection limit is necessary, or if a complex heterogeneous class of side products or impurities, like host cell proteins, must be removed to a level below 10 ppm, based on the mass of the final API.

The use of any composite material of any of the below or above embodiments in a sequence with any other purification steps, is therefore subject to the present invention.

In combination with any of the above or below embodiments, their use either before or after an ion exchanger or affinity chromatography step, or any other purification steps is within the scope of the present invention, particularly if affinity based separation steps, e.g. selective adsorption of target compounds to any kind of separation media harbouring protein A, protein G, or a combination of both is considered.

Other purification steps are comprising composite materials of the present invention in combination with any of the above and below embodiments.

In addition, any combination with membrane filtration, depth filtration or applying a monolithic separation agent, is considered within the scope of the present invention. In a more preferred embodiment, in combination with any of the above or below embodiments, the polymeric mesh is used before or after an ion exchanger or affinity chromatography step, or other purification steps.

In one preferred embodiment, in combination with any of the above or below embodiments, said ion exchanger material is a composite material of the present invention, more preferred a composite bearing at least one amino, carboxyl or sulphonyl residue, most preferred, a primary amine or a reaction product of a polymer comprising maleic anhydride units.

Accordingly, the present invention is related to a combination with one or more additional separation steps, characterized in that the above steps i), ii), and iii) are carried out with the raw feed suspension or solution, in advance to any further chromatographic or non chromatographic purification step.

Materials and Methods

Support Material

Silica Gel Davisil LC 250 (W.R. Grace), average nominal pore size 250 Å, particle size 40-63 μm

Polymers

Poly(vinylformamid-co-polyvinylamin) solution in water, Lupamin 45-70 (BASF) supplier: BTC Europe, Monheim, Germany, degree of hydrolysis about 30% according to the information obtained by the supplier.

Poly(methyl vinyl ether-alt-maleic anhydride), (Sigma-Aldrich, Steinheim, Germany)

Cross-Linker

Hexanediol diglycidylether, Ipox RD 18, ipox chemicals, Laupheim (Germany).

Poly(ethyleneglycol)diglycidyl ether M=500 Da, (Sigma-Aldrich, Steinheim, Germany)

Proteins

Albumin, IgG-frei <98%, and Lysozym, min. 35000 FIP U/mg, (both by Carl Roth, Karlsruhe, Germany)

Polyclonal hlgG Octagam solution, 5mg/ml (Octapharma, Heidelberg, Germany)

Reagents

Alizarin S (Carl Roth, Karlsruhe, Germany)

Any other chemical and solvents from Sigma-Aldrich, Steinheim, Germany

Pore size distribution and pore volume fractions of the various composite adsorbents

The accessible pore volume fractions, which are correlated to the pore diameters and the exclusion limits for polymer molecules with various hydrodynamic radius have been determined using inverse Size Exclusion Chromatography (iSEC). For this purpose, the composite material is preferably packed into a 1 ml (50×5 mm) chromatographic column, equilibrated with 20 mM aqueous ammonium acetate buffer at a pH between 6 and 9, and calibrated by applying two low molecular weight standards, and a selection of commercial pullulane polymer standards of known defined average molecular weights Mw (PPS, Mainz Germany).

The molecular weight determination of the pullulane standards was achieved at PSS by SEC with water, sodium azide 0.005% as mobile phase at a flow rate of 1 ml/min at 30° C. Three analytical columns, each 8×300 mm (PSS SUPREMA 10μm 100 Å/3000 Å/3000 Å), have been used in in-line combination with an 8×50 mm pre-column (PSS SUPREMA 10 μm). Sample concentration was 1 g/l, injected volume 20 μl in each run. Detection was achieved with a refractive index (RI) monitor (Agilent RID), connected to a PSS WinGPC Data Acquisition system.

The pore volume fraction Kₐᵥ, accessible for the particular standards in a particular composite material, was obtained by evaluation of the net elution volume Vₑₙ (μl).

Accordingly, Kₐᵥ describes the fraction of the overall pore volume, a particular standard with given hydrodynamic radius Rₕ can access. Methanol is used for the determination of the total liquid volume Vₜ=Vₑ=V₀ representing a Kₐᵥ value of 1. The pullulane standard of 210,000 Da is used to determine the interstitial volume Vᵢ, between the packed composite particles, representing the liquid volume outside the particles, as it is already excluded from the pores, thus representing a Kₐᵥ of 0 (0% of the pore volume). The difference between V₀ and Vᵢ is the pore volume Vₚ.

The following SEC Standards are preferably used:

Methanol
Ethylene glycol

| | | | | | |
|---|---|---|---|---|---|
| Pullulan | 6.2 | kD | Rₕ | 2.13 | nm |
| Pullulan | 10.0 | kD | Rₕ | 2.70 | nm |
| Pullulan | 21.7 | kD | Rₕ | 3.98 | nm |
| Pullulan | 48.8 | kD | Rₕ | 5.96 | nm |
| Pullulan | 113 | kD | Rₕ | 9.07 | nm |
| Pullulan | 210 | kD | Rₕ | 12.37 | nm |

The partial pore volumes are defined as the respective volume fractions in the composite adsorbent, which can be accessed by not retained pullulane polymer standards, as well as by not retained smaller molecules. Not retained means, that in order to determine only the pore volume fractions, no interaction or binding of the respective standard occurs on the surface of a stationary phase. For the support material and the composites of the present invention this is the case for alcohols and hydrophilic carbohydrates, preferably pullulanes, exhibiting known hydrodynamic radii (Rₕ) in aqueous solvent systems.

The Rₕ values of the pullulanes have been calculated from the molecular weight Mw according to the empiric equation Rₕ=0.027 Mw^{0.5} (I.Tatarova et al., J. Chromatogr. A 1193 (2008), p.130).

The Rₕ value of IgG was taken from the literature (K. Ahrer et al., J. Chromatogr. A 1009 (2003), p. 95).

Thermogravimetry

TGA 2 Star System, Mettler Toledo, (Gießen, Germany)

Air flow 40 ml/min, nitrogen pressure 1 bar.

Temperature gradient: Start temperature 30° C. Heating rate 25° C/min.

Segment 1: From 30° C. to 120° C. in 3.6 min;

Segment 2: Hold at 120° C. for 15 min;

Segment 3: From 120° C. to 130° C. in 0.4 min;

Segment 4: Hold at 130° C. for 15 min;

Segment 5: From 130° C. to 350° C. in 8.8 min;

Segment 6: Hold at 350° C. for 15 min;

Segment 7: From 350° C. to 1000° C. in 26 min.

The weight loss of the composite material within the temperature interval between 130° C. and 720° C. is the measure for the amount of the polymeric coating.

The sample starting weight was between 15 mg and 25 mg. Samples have been dried in advance at 105° C., 200 mbar.

UV Measurement

Specord 50, Analytik Jena (Jena, Germany)

Measurement of the adsorption at 254 nm and 280 nm. The adsorption values at 280 nm were used for the determination of the protein binding and recovery rates.

### EXAMPLES

Adsorbent transfer from aqueous to water-free solvents and reverse. Any derivatisation and cross-linking of polymers using reagents easily hydrolising with protic solvents must be carried out in water-free solvents, which will in addition allow, a satisfactory swelling of the polymer. Dimetyl formamide (DMF) is preferred for these purposes. Alternatively N-methyl pyrrolidone, dimethyl acetamide and dimethyl sulfoxide may be used.

In order to remove and exclude water, a careful stepwise solvent transfer of the composite materials is critical, applying preferably the following repetitive steps:

During each step the resin is suspended with three bed volumes of the particular solvent, stirred during five minutes, and then suction-dried. With each solvent the wash is repeated five times. The first solvent is dry ethanol, followed by dry acetone, and finally DMF.

Using epoxide reagents subsequently, a water content of less than 0.05% in the particular solvent is sufficient. Using very sensitive reagents like carbonyl diimidazole (CDI), 100 ppm of water should be not exceeded.

The transfer from DMF back to aqueous solvents preferably includes a wash with three bed volumes of either ethanol or methanol or acetone five times.

### Reference Example 1

Preparation of a Cross-Linked Poly (Vinylamine) Del

In order to check the reaction without support material, 3 ml of the polymer—cross-linking agent solution of Example 1 was heated for 24 hours at 60° C. After six hours the gelation was visible. After 24 hours one piece of a transparent solid elastic gel was obtained.

### Example 1

Composite Material Containing Cross-Linked Poly(Vinylamine) 1.5 ml of hexane diol diclycidylether (Mw 230.2, d=1.07 g/ml) cross-linker were shaken with 90 ml water, forming a homogeneous emulsion. This cross-linker solution was added to 30 ml of an aqueous solution of poly(vinylformamid-co-polyvinylamin) (Lupamin 45-70, raw and untreated). After mixing, the pH was 9.5. 28 g (100 ml dry sedimentation volume) of Silica Gel Davisil LC 250 40-63 μm, dry powder, were sedimented into a flat bottom stainless steel dish with 15 cm diameter. The bed height was about 25 mm. 110 ml of polymer-cross-linker solution were added in portions, equally distributed over the silica, whereas the solution was soaked in the pores, forming a viscous, mucous mass. Shaking the dish during 10 min. at 200 rpm using a gyratory shaker, the mixture was homogenized. After adding a solution of 10 ml of diluted polymer (2.5 ml of poly(vinylformamide-co-polyvinylamine) diluted with 7.5 ml of water) the suspension became smooth. The resultant paste was covered by a liquid film of about 2 mm height. After closing the dish with a stainless steel lid, the batch was heated without further mixing or moving for 24 hours in a drying oven at 60° C. yielding the moist composite.

The product cake was washed five times with three bed volumes of water on a funnel with sintered G3 frit. During each washing step the solid material was thoroughly suspended and gently stirred to obtain a homogeneous suspension.

Finally the suspension was aspirated and ready to use in reactions according to Example 3.

### Example 1a

Composite Material Containing Cross-Linked Poly(Vinylamine) Hydrochloride.

After the fifth aspiration step of the composite material of Example 1 three bed volumes of 2 M hydrochloric acid were added and the suspension was stirred for 10 min. Subsequently the product cake was washed three times with always three bed volumes of water, once with methanol: water 50:50, and three times with water again, always on a funnel with sintered G3 frit. The pH of the final eluate was 6.

After careful aspiration the composite material was dried at 105° C. and 200 mbar for 18 hours yielding an off-white powder.

The nitrogen content was determined to 2.74%, the carbon content to 6.57% and chlorine to 4%, each w/w.

In order to completely hydrolyse eventually unreacted epoxy groups, 100 ml of the composite material may be further shaken with 200 ml of 2 n hydrochloric over two hours at ambient temperature.

### Example 2

Preparation of a Phenyl Resin by Derivatisation of a Composite Containing Poly(Vinylamine) with Benzoic Anhydride.

The following operations were carried out at a temperature between 19° C. and 22° C.

Using a funnel with sintered G3 frit, 5.3 g of the composite material of Example la, containing cross-linked poly(vinylamine) hydrochloride, was transferred into a water-free environment as described above, suspended in dimethyl formamide (DMF), and suction dried.

280 mg benzoic anhydride (Mr=226, 1.24 mmol) were dissolved in 16 ml DMF, mixed with the composite in a round bottom flask, and gently shaken for 15 min, in order to achieve an equal distribution of the reagent inside the support pores. The liquid was then aspirated.

140 mg (0.62 mmol) of benzoic anhydride were dissolved in 15 ml of a 1 M triethylamine (Mr=101) solution in DMF. This solution was mixed with the moist filter cake of the composite material, and the suspension was shaken for two hours using a gyratory shaker at about 200 rpm. After aspiration the resin cake was washed two times with 30 ml of DMF, two times with 30 ml acetone, and two times with 30 ml water. Subsequently the amino groups of the composite material were protonated digesting the resin with 30 ml of 1 M hydrochloric acid for 10 min. The solution was aspirated, washed four times with water until the pH of the eluate was between 5 and 6, and then suction dried.

After drying at 105° C. at reduced pressure (200 mbar) for 12 hours 6.7 g of the off-white composite product were obtained.

### Example 3

Preparation of a Carboxyphenylamide Resin by Derivatisation of a Composite Containing Poly(Vinylamine) with Phthalic Anhydride.

The following operations were carried out at a temperature between 19° C. and 22° C.

Using an extraction tube (Macherey Nagel, empty Chromabond Column with PE frit), 7.5 ml of the polv(vinvlamine) composite according to Example 1 were protonated with a solution of 50 mM ammonium acetate, pH 6, transferred into a water-free environment as described above, suspended in dimethyl formamide (DMF), and suction dried. The composite was then mixed with 7.5 ml of 1 M phthalic anhydride (7.5 mmol, Mr=148) solution in DMF, and gently shaken for 15 min, in order to achieve an equal distribution of the reagent inside the support pores. The liquid was then aspirated. 7.5 ml of 1 M triethylamine (Mr=101) in DMF were added and the suspension was shaken for three hours at 20° C. using a gyratory shaker at about 200 rpm. After aspiration the resin cake was washed with 7.5 ml of DMF.

Subsequently 7.5 ml of 2 M triethylamine in DMF were added and the mixture shaken as before, thus ensuring that the residual amino groups and carboxyl groups will stay deprotonated, and aspirated again.

Then another 7.5 ml of 1 M phthalic anhydride were added, shaking the suspension again for three hours, which was finally aspirated, and the filter cake washed three times with each 7.5 ml DMF.

For the final purification the product was washed at least twice in series, each with acid followed by base (to induce alternating shrinking and swelling of the polymer layers) in DMF (not water because the composite is rather lipophilic), in order to rinse out potentially entrapped traces of polymer derivatives which may have not been covalently immobilized. Suitable acids for this purpose are toluene sulfonic acid or trifluoro acetic acid and triethylamine as a base at concentrations of 100 mM.

The above composite is rather lipophilic. For several application purposes it is recommended to reduce the carboxy phenyl concentration or to mix or even replace this ligand with short chain carboxylates, e.g., with succinic anhydride or iso valeric anhydride as starting materials.

### Example 4

Preparation of a Carboxyethyl Resin by Derivatisation of a Composite Containing Poly(Vinylamine) with Succinic Anhydride.

The following operations were carried out at a temperature between 19° C. and 22° C.

Using a funnel with sintered G3 frit 8 g of the composite material of Example 1a, containing cross-linked poly(vinylamine) hydrochloride, were transferred into a water-free environment as described above, suspended in dimethyl formamide (DMF), and suction dried.

5 g succinic anhydride (50 mmol, Mr=100) were dissolved in 25 ml DMF, mixed with the composite in a round bottom flask, and gently shaken for 15 min, in order to achieve an equal distribution of the reagent inside the support pores. The liquid was then aspirated. 5 g (50 mmol) of succinic anhydride were dissolved in 25 ml of a 2 M triethylamine (Mr=101) solution in DMF. This solution was mixed with the moist filter cake of the composite material, and the suspension was shaken for three hours using a gyratory shaker at about 200 rpm.

After aspiration the resin cake was washed five times with 75 ml of DMF and then exposed while gently stirring to a solution of 2 M triethylamine in DMF for 15 min, thus ensuring that the residual amino groups and carboxyl groups will stay deprotonated, and aspirated again.

Then another 25 ml of 1 M succinic anhydride were added, shaking the suspension again for three hours, which was finally aspirated, and the filter cake washed three times with DMF, twice with methanol, and five times with water, each 75 ml.

Subsequently the carboxylic groups in the composite material were converted into the protonated form digesting with 1 M hydrochloric acid for 15 min. The solution was aspirated, washed four times with water until the pH of the eluate was between 5 and 6, and then suction dried.

After drying at 105° C. at reduced pressure (200 mbar) 9.4 g of the composite product were obtained.

### Example 5

Two-Phase Reaction, Neutralizing the Poly(Vinylamine) Composite Amino Groups at the Particle Outer Surface (Rim) by Acetylation.

10 ml of acetic anhydride and 5 ml triethylamine were dissolved in 50 ml toluene. 13.5 g of water containing, moist composite material (equivalent to 4 g of dry material) of Example 1a was mixed with the reagent solution in toluene.

The composite wettability was initially poor. After 10 min of vigorous shaking a homogeneous opaque suspension was obtained

The reaction was stopped after 20 min by aspiration, suspending and gently stirring the filter cake in three bed volumes of acetone for 5 min, and subsequently washing the filter cake with three bed volumes of acetone, methanol, and water, until the pH in the supernatant was 7.5.

A sample of the last filter cake was contacted with an aqueous solution of alizarin S (10 mM), exhibiting a bright orange colour.

The solids remained white for 20 seconds, before the colour gradually changed to pink, and finally to reddish.

In contrast, the starting material of Example 1a became immediately deep violet with Alizarin S, significantly darker compared to the acetylated product.

Thermogravimetry: Weight loss 17.6%

### Example 6

Composite Material Containing Cross-Linked Poly(Vinylformamid-Co-Polyvinylamin).

1.15 ml of poly(ethyleneglycol) diglycidyl ether (average Mw 500, d=1.14 g/ml) cross-linker were mixed with 45 ml water, forming a clear solution. This cross-linker solution was added to 15 ml of an aqueous solution of poly(vinylformamid-co-polyvinylamin) (Lupamin 45-70, raw and untreated). After mixing, the pH was 9.5.

14 g (50 ml bulk volume) of Silica Gel Davisil LC 250, 40-63 μm, dry powder, were filled into a flat bottom stainless steel dish with 12 cm diameter. The plain bed height was about 20 mm.

55 ml of polymer cross-linker solution were added in portions, equally distributed over the silica, whereas the solution was soaked in the pores, forming a viscous and mucous mass. The mixture was homogenized by shaking the dish for 10 min at 200 rpm, using a gyratory shaker. After adding a solution of 2 ml of diluted polymer (2.5 ml of poly(vinylformamide-co-polyvinylamine) and 7.5 ml of water) the suspension became smooth. The resultant paste remained covered with a liquid film of about 2 mm height. After closing the dish with a stainless steel lid, the mixture was heated without further mixing or moving for 4 hours in a drying oven at 80° C. yielding the moist composite.

The product cake was digested two times with four bed volumes of water, and the supernatant was disposed after sedimentation. The residue was then suspended in 2 N hydrochloric acid and gently stirred for 10 min. The supernatant was disposed after sedimentation. This procedure was repeated three times with water, once with 0.6 M sodium carbonate, and again three times with water. The aqueous suspension was subsequently aspirated on a funnel with sintered G4 frit, digested in 2 N hydrochloric acid for 5 min, and washed three times with four bed volumes water, until the pH was 5.

Finally the suspension was aspirated, the filter cake collected and dried at 105° C. in vacuo (200 mbar) during four hours, to obtain the composite material ready to use in applications according to Examples 8-11.

Thermogravimetry: Weight loss 15.7%

### Reference Example 6

Preparation of a Cross-Linked Poly(Vinylamine) Gel

In order to check the reaction without support material, 3 ml of the polymer—cross-linking agent solution of Example 6 was heated for 4 hours at 80° C. After one hour the gelation was complete. After 2 hours one piece of a transparent solid elastic gel was obtained.

### Example 7

Synthesis of a Carboxylic Groups Containing Composite Material Starting from a Maleic Anhydride Co-Polymer.

84 mg (724 μmol, M=116) of hexamethylene diamine were dissolved in 50 ml methanol and converted into the hydrochloride by adding 210 μl (1470 μmol) of 7 N hydrochloric acid.

32.5 ml of this cross-linker solution was added to 10 g of Silica Gel Davisil LC 250, and the solvent was evaporated by warming to 30° C. The cross-linker hydrochloride remained in the silica gel pores.

2.7 g of poly(methyl vinyl ether-alt-maleic anhydride) were dissolved with 20 ml of acetone at 20° C., and 515 pl of triethylamine (3.4 mmol, M=101, D=0.726), were added, yielding a clear red solution.

This solution was poured over the silica gel impregnated with the cross-linker. The polymer solution was rapidly soaked by the silica, whereas the colour of the wetted solid turned to bright red. The interstitial volume between the silica particles remained only slightly wetted.

After 10 min the wet cake was suspended in 100 ml of acetone, and the suspension aspirated using a funnel with sintered G4 frit. The filtrate was initially pink, the filter cake became slightly pinkish after washing with each three bed volumes of acetone, and remained white after a two times methanol wash with three bed volumes.

Hydrolysis of the Remaining Anhydride Groups.

1.65 g (27 mmol, M=61, D=1.02) of ethanolamine were dissolved in 20 mL of methanol. This solution was added to the above moist maleic anhydride coated silica and allowed to react for 20 min.

This suspension was aspirated, washed with three bed volumes of methanol, digested with 2 N aqueous hydrochloric acid over 5 min., washed with water, until the pH became 5, and finally washed with methanol.

After drying for 18 hours at 60° C. and 150 mbar the colour of the powder was off-white.

Thermogravimetry: Weight loss 12.6%

After incubating with 200 mM aqueous sodium carbonate solution, pH 9, for 5 min. aspiration, and washing three times with five bed volumes of water the composite material Example 7 was dried for 4 hours at 105° C. and subsequently used in applications according to Examples 8-11

Protein Binding Studies

One basic protein, lysozyme with a pl of 11.3, and one acidic protein, albumin with a pl of 4.9, were chosen for the binding studies, whereas the polyclonal hlgG (Octagam) used is a mixture of numerous entities with pl values spreading from 4 to 10.

The binding and recovery experiments were performed in the batch mode utilizing pure solutions of individual proteins, as well as with mixtures of albumin and lysozyme.

The recovery rate of hlgG was calculated from the UV absorption values of the recovered supernatant and the feed solution.

The binding rates of albumin and lysozyme were calculated from the difference of the UV absorption values between the feed solution and the recovered supernatant. The UV calibration was carried out at 280 nm with protein concentrations between 1 mg/ml and 30 pg/ml.

Batch Binding/Size-Exclusion Experiments with Single Adsorbent-Protein Combinations.

The proteins were dissolved in 50 mM aqueous ammonium acetate solution at pH 6.5. The pH was adjusted using 2 M acetic acid. The resultant pH of these protein solutions was between 6 and 7. The adsorption of the proteins albumin and lysozyme, and size-exclusion of hlgG were investigated at protein concentrations between 3 mg/ml and 0.5 mg/ml with the composite materials of Examples 6 and 7.

The mass-to-volume-ratios between adsorbent and feed solution were between 1:13 and 1:40 (1 g adsorbent and 13 ml, respectively 40 ml of feed).

### Example 8

100 mg of the dry adsorbent were wetted with 200 μl of 50 mM aqueous ammonium acetate solution, then incubated with 4 ml of the protein solution (c=0.5 mg/ml), and contacted over 10 min, while gently shaken. The supernatant was removed after sedimentation and filtrated through a 20 μm polyether sulfone (PES) membrane. This filtrate was used for determination of the protein concentration by measurement of optical density (UV, 280 nm).

Results

A 96% recovery rate of hlgG was determined with the adsorbent of Example 7, made from poly(methyl vinylether-a/t-maleic anhydride) equipped with carboxylic and hydroxyethyl groups, starting from an 0.5 mg/ml initial hlgG concentration. A 95% binding rate of lysozyme was determined with this adsorbent, starting from an 0.5 mg/ml initial concentration.

A 100% recovery rate of hlgG was determined for the basic adsorbent of Example 6, made from poly(vinylformamide-co-polyvinylamin) equipped with amino groups and formyl groups, starting from an 0.5 mg/ml initial hlgG concentration. A 85% binding rate of albumin was determined with this adsorbent, starting from an 0.5 mg/ml initial concentration.

### Example 9

300 mg of the dry adsorbent of Example 6 were wetted with 800 μl of 50 mM aqueous ammonium acetate solution, then incubated with 4 ml of the protein solution (mg/ml), and contacted over 10 min, while gently shaken. The supernatant was removed after sedimentation and filtrated through a 20 μm polyether sulfone (PES) membrane. The remaining solution was used for the measurement of the optical density (UV, 280 nm) of the protein concentration.

From 12 mg albumin offered, 11.6 mg were bound (96.5%), equal to 39 mg per gram of adsorbent.

The following examples were carried out with combinations of two composite adsorbents, used for protein binding in the presence of 150 mM salt.

### Example 10

Mixture of Equal Amounts of Two Adsorbents, One Comprising Amino Groups and the other Comprising Carboxylic Groups.

The protein solution contained 0.5 mg/ml albumin and 0.25 mg/ml lysozyme, dissolved in 50 mM aqueous ammonium acetate solution, 100 mM sodium chloride. Each 200 mg of the dry adsorbent according to Examples 6 and 7 were mixed and wetted with 800 pl of an 50 mM aqueous ammonium acetate solution containing 100 mM sodium chloride, then incubated with 8 ml of the protein solution, and contacted over 15 min, while gently shaken. After sedimentation, the supernatant was removed and filtrated through a 20 μm polyether sulfone (PES) membrane. This filtrate was used for the measurement of the optical density (UV, 280 nm) of the protein concentration.

A total 95% depletion rate of the proteins was observed.

Example 11

Depletion applying two subsequent steps. Means applying the amino group containing adsorbent of Example 6, followed by the carboxylic group containing adsorbent of Example 7.

The concentration of the protein solution was 0.5 mg/ml albumin and 0.25 mg/ml lysozyme, dissolved in 50 mM aqueous ammonium acetate solution, 100 mM sodium chloride.

200 mg of the dry basic adsorbent of Example 6 was wetted with 400 μl of an 50 mM aqueous ammonium acetate solution containing 100 mM sodium chloride, then incubated with 6 ml of the protein solution, and contacted over 15 min, while gently shaken. The supernatant was removed after sedimentation, and 4 ml were contacted with 100 mg of the acidic adsorbent of Example 7 and gently shaken for 15 min.

After sedimentation, the supernatant was filtrated through a 20 μm polyether sulfone (PES) membrane. This filtrate was used for the measurement of the optical density (UV, 280 nm) of the protein concentration.

An overall 90% depletion of the proteins was determined after these two subsequent steps.

In another aspect, the invention relates to the following items:
1. A composite material comprising a support material and at least one polymeric layer, wherein the at least one polymeric layer is present in form of a polymeric mesh and is comprising at least one non-adsorbing/non-adsorptive polymer, characterized in that the polymeric layer is adapted such that the at least one non-adsorbing/non-adsorptive polymer of said polymeric layer is capable of coming into contact with a liquid phase.
2. The composite material according to item 1, wherein the composite material or the polymeric mesh exhibits a pore size exclusion limit for a target compound with a hydrodynamic radius Rₕ of from 1 nm to 12 nm, as calibrated with pullulan standards exhibiting a hydrodynamic radius Rₕ.
3. The composite material according to item 1 or 2 comprising at least one first polymeric layer comprising at least one non-adsorbing/non-adsorptive polymer and at least one second polymeric layer comprising at least one adsorbing/adsorptive polymer, wherein said at least one first polymeric layer is present at the outermost surface of the outermost second polymer layer.
4. The Composite material according to item 2 or 3 wherein the pore size exclusion limit for target compounds with a hydrodynamic radius Rₕ of from 1 nm to 12 nm, is calibrated in 20 mM ammonium acetate buffer at a pH between 5 and 9 with inverse size exclusion chromatography (iSEC) of pullulan standards exhibiting a molecular weight between 1,000 Da and 210,000 Da.
5. The composite material according to any of the preceding items, wherein the at least one non-adsorbing/non-adsorptive polymer comprises (at least one) polar residue selected from hydroxyl (OH—), diol, methyloxy (—O—CH₃), formyl-, acetyl-, primary or secondary amide, or ethylene oxy-.
6. The composite material according to any of the preceding items, wherein the at least one non-adsorbing/non-adsorptive polymer is selected from poly(vinyl formamide), poly(vinyl acetamide), poly(vinyl pyrrolidone), poly(vinylalcohol), poly(vinylacetate), poly(ethyleneglycol), poly(hydroxyethyl acrylate), poly(hydroxyethyl methacrylate), poly(acrylamide), poly(methacrylamide), amylose, amylopektin, agarose, any kind of hydroxylmethyl celluloses, hydroxyethyl celluloses, hydroxypropyl celluloses, hydroxypropyl methyl cellulose, methylcellulose, acetylcellulose, or mixtures thereof.
7. The composite material according to any of items 3 to 6, wherein said at least one adsorbing/adsorptive polymer comprises either at least one anionic, or at least one cationic, or at least one lipophilic or at least one hydrophilic residue, or combinations of two, three or four different species/kinds of said residues.
8. The composite material, or a composite material according to any of the preceding items, wherein the at least one adsorptive/adsorbing polymer comprises poly(maleic anhydride) building blocks/monomer units, which are comprising in turn precursor ligands for anionic and lipophilic or hydrophilic residues.
9. The composite material according to item 8, wherein the at least one adsorptive/adsorbing polymer comprises hydrolysed poly(maleic anhydride) monomer units, which comprise in turn precursor ligands for anionic and lipophilic or hydrophilic residues.
10. A combination of at least one first adsorbent comprising the at least one composite material according to any of the preceding items, in particular according to item 3, and at least one second adsorbent, wherein said second adsorbent is different from said first adsorbent, in particular wherein said first and second adsorbent both comprise at least one composite material according to any of the preceding items, which are different from each other, or wherein said first adsorbent comprises the at least one composite material according to any of the preceding items and said second adsorbent is an inorganic adsorbing material.
11. A system comprising at least one composite material of any of the preceding items and at least one liquid phase, wherein the at least one composite is equilibrated with said liquid phase.
12. Method for the selective derivatisation of the functional groups of a polymer comprised by a polymeric mesh, characterized in that the polymeric mesh is filled with a solution of a derivatisation reagent in a solvent which is water-immiscible, wherein the volume outside of this mesh is filled with an aqueous solvent.
13. Method for the selective derivatisation of the functional groups of a polymer comprised by a polymeric mesh, characterized in that the polymeric mesh is filled with a solution of a derivatisation reagent in a solvent, preferably an aqueous or organic solvent, wherein the volume outside of this mesh is empty/not containing a liquid.
14. Method for the selective derivatisation of the functional groups of a polymer comprised by a polymeric mesh, characterized in that the polymeric mesh is filled with an aqueous solvent, wherein the volume outside of this mesh, but in contact with its boundary surface, is filled with a solution of a derivatisation reagent in a solvent which is water-immiscible.
15. A method for recovering a target compound from a feedstock, said feedstock being in the form of a solution or suspension, and being preferably a fermentation broth, and comprises at least one target compound, preferably a protein, more preferably an antibody and at least one impurity compound, preferably selected from host cell proteins (HCP), DNA, RNA or other nucleic acid, or a combination of two or more thereof, and optionally comprising albumins, endotoxins detergents and microorganisms, or fragments thereof, or a combination of two or more thereof, said method comprising the steps of:
   i) contacting said feedstock with at least one composite adsorbent according to any of the preceding items for a sufficient period of time, wherein at least one impurity compound is retained;
   ii) subsequently, separating the at least one composite adsorbent from the purified feedstock containing at least one target compound;
   iii) optionally, isolating the target compound from the feedstock.
16. The method for recovering a target compound from a feedstock according to item 15, wherein the target compound is a target protein being characterized by a hydrodynamic radius Rₕ₁ and the impurity compound being characterized by a hydrodynamic radius Rₕ₂, wherein Rₕ₁>Rₕ₂, the method comprising the following steps (i) to (iv) and optionally step (v):
   (i) providing at least one composite adsorbent exhibiting a pore size exclusion limit Rₕᵢ which can be set variably;
   (ii) adapting the variable pore size exclusion limit Rₕᵢ of the at least one composite adsorbent to the hydrodynamic radii Rₕ₁ and Rₕ₂ such that R₂<Rₕᵢ and Rₕ₁>Rₕᵢ;
   (iii) contacting the at least one composite adsorbent with the feedstock for a time sufficient to allow retaining the impurity compound in the at least one composite adsorbent and excluding the target protein from the at least one composite adsorbent;
   (iv) separating the at least one composite adsorbent containing the retained impurity compound from the feedstock containing the excluded target protein in order to obtain a purified feedstock;
   (v) optionally, isolating the target protein from the purified feedstock.
17. The method for recovering a target compound from a feedstock according to item 15 or 16, wherein the target compound is a target protein being characterized by a hydrodynamic radius Rₕ₁ and the impurity compound being characterized by a hydrodynamic radius Rₕ₂, wherein Rₕ₁>Rₕ₂, the method comprising the following steps (i) and (iii) to (iv) and optionally step (v):
   (i) providing the at least one composite adsorbent comprising at least one polymer, the at least one composite adsorbent being characterized by a pore size exclusion limit Rₕᵢ such that Rₕ₂<Rₕᵢ and Rₕ₁>Rₕᵢ;
   (iii) contacting the at least one composite adsorbent with the feedstock for a time sufficient to allow retaining the impurity compound in the at least one composite adsorbent and excluding the target protein from the at least one composite adsorbent;
   (iv) separating the at least one composite adsorbent containing the retained impurity compound from the feedstock containing the excluded target protein in order to obtain a purified feedstock;
   (v) optionally, isolating the target protein from the purified feedstock.
18. Method of item 16 or 17, wherein said setting in step (i) or said adapting in step (ii) or said setting in step (i) and said adapting in step (ii) is performed by one or more of the following: varying the structure of the polymer, selecting the cross-linker used to generate a crosslinked polymer, selecting the degree of cross-linkage of the polymer, controlling the degree of swelling of the polymer by varying the solvent for the preparation and the use of the polymer, particularly varying the pH of the solvent and thus the degree of protonation of the polymer, and controlling the concentration and the immobilized amount of the polymer within said adsorbent composite.

1. A composite material for recovering a target compound from a solution or suspension,
the composite material comprising a support material and a polymeric mesh, said polymeric mesh comprises at least one polymeric layer and
comprises at its outermost surface at least one polymer which is non-adsorbing/non-adsorptive for the target compound,
wherein the at least one non-adsorbing/non-adsorptive polymer is capable of coming into contact with said solution or suspension; and
wherein said solution or suspension comprises said target compound and further comprises at least one impurity compound, and said composite material further comprises sites which are adsorbing/adsorptive for said at least one impurity compound.

2. The composite material of claim 1, wherein said polymeric mesh comprises at least one first polymeric layer and at least one second polymeric layer, the at least one first polymeric layer is at the outermost surface of the polymeric mesh and comprises at least one polymer which is non-adsorbing/non-adsorptive for the target compound, and the at least one second polymeric layer comprises at least one polymer which is adsorbing/adsorptive for the at least one impurity compound, wherein said at least one first polymeric layer is present at the outermost surface of the at least one second polymeric layer.

3. The composite material of claim 1, wherein the adsorbing/adsorptive sites consist of the surface of the support.

4. The composite material according to claim 1, wherein the at least one non-adsorbing/non-adsorptive polymer comprises a polar residue selected from hydroxyl (OH—), diol, methyloxy (—O—CH₃), formyl-, acetyl-, primary and secondary amide, ethylene oxy-, and a combination of two or more of said residues.

5. The composite material according to claim 1, wherein the at least one non-adsorbing/non-adsorptive polymer is selected from poly(vinyl formamide), poly(vinyl acetamide), poly(vinyl pyrrolidone), poly(vinyl alcohol), poly(vinylacetate), poly(ethyleneglycol), poly(hydroxyethyl acrylate), poly(hydroxyethyl methacrylate), poly(acrylamide), poly(methacrylamide), amylose, amylopektin, agarose, any kind of hydroxylmethyl celluloses, hydroxyethyl celluloses, hydroxypropyl celluloses, hydroxypropyl methyl cellulose, methylcellulose, acetylcellulose, copolymers of said polymers, and a combination of two or more thereof.

6. The composite material according to claim 1,
wherein poly(maleic anhydride) or a co-polymer thereof is immobilized on said support, either cross-linked or covalently attached to said support, wherein said poly(maleic anhydride) or a co-polymer thereof comprises precursor ligands for anionic and lipophilic or hydrophilic residues.

7. The composite material of any claim 2, wherein the at least one adsorptive/adsorbing polymer comprises poly(maleic anhydride) building blocks/monomer units, which comprise precursor ligands for anionic and lipophilic or hydrophilic residues.

8. The composite material according to claim 6, wherein the at least one adsorptive/adsorbing polymer comprises hydrolysed poly(maleic anhydride) monomer units, which comprise anionic and lipophilic or hydrophilic residues.

9. A combination of at least one first adsorbent and at least one second adsorbent, wherein the at least one first adsorbent is equal to the at least one second adsorbent or is different therefrom,
the at least one first adsorbent comprises at least one composite material, the composite material comprising at least one adsorbing/adsorptive polymer, or at least one non-adsorbing/non-adsorptive polymer, or at least one adsorbing/adsorptive polymer together with at least one non-adsorbing/non-adsorptive polymer.

10. The combination of claim 9, wherein the at least one second adsorbent comprises at least one composite material, the composite material comprising at least one adsorbing/adsorptive polymer, or at least one non-adsorbing/non-adsorptive polymer, or at least one adsorbing/adsorptive polymer together with at least one non-adsorbing/non-adsorptive polymer.

11. The combination of claim 9, wherein the at least one second adsorbent is an inorganic adsorbent or an ion-exchange material.

12. The combination of claim 9, wherein said at least one first adsorbent and the at least one second adsorbent are provided in at least two separate means respectively configured to perform in chromatography, in a fluidized bed, in an expanded bed, or in a batch separation.

13. The combination of claim 9, wherein the combination is a mixture of the at least one first adsorbent and the at least one second adsorbent, wherein said mixture is provided either in a chromatographic column or in means configured to perform in a fluidized bed, in an expanded bed, or in a batch separation.

14. The combination of claim 13, wherein the mixture is applied in combination with another mixture of at least one first and at least one second adsorbent;
wherein the combination respectively is provided in at least two separate means configured to perform in chromatography, in a fluidized bed, in an expanded bed, or in a batch separation.

15. The composite material as defined in claim 1,
wherein at least one adsorbing/adsorptive polymer and/or at least one non-adsorbing/non-adsorptive polymer is/are derivatized.

16. The composite material according to claim 1, wherein said at least one adsorbing/adsorptive polymer comprises either at least one anionic, or at least one cationic, or at least one lipophilic or at least one hydrophilic residue, or a combination of two, three or four different species/kinds of said residues.

17. The composite material according to claim 1, wherein the composite material or at least one polymeric layer of the polymeric mesh exhibits a pore size exclusion limit for a target compound with a hydrodynamic radius Rh of from 1 nm to 12 nm, as calibrated with pullulane standards exhibiting a hydrodynamic radius Rh.

18. The composite material according to claim 17, wherein the pore size exclusion limit for a target compound or target compounds with a hydrodynamic radius Rh of from 1 nm to 12 nm, is calibrated in 20 mM ammonium acetate buffer at a pH between 5 and 9 with inverse size exclusion chromatography (iSEC) of pullulane standards exhibiting a molecular weight between 1,000 Da and 210,000 Da.

19. A system comprising at least one composite material as defined in claim 1, and at least one liquid phase.

20. A method for recovering a target compound from a solution or suspension, either being a feedstock, or being the eluate of a preceding purification step, said solution or suspension comprises said target compound, and further comprises at least one impurity compound, said method comprising the steps of:
i) contacting said solution or suspension with at least one composite material according to claim 1, for a sufficient period of time, wherein said at least one impurity compound is retained;
ii) subsequently, separating the at least one composite material from the purified solution or suspension containing the target compound;
iii) optionally, isolating the target compound from the solution or suspension.

21. The method according to claim 20, contacting said feedstock in step (i) with at least one adsorbent of a combination comprising at least one first adsorbent and at least one second adsorbent wherein the at least one first adsorbent is equal to the at least one second adsorbent or is different therefrom, the at least one first adsorbent comprises at least one composite material, the composite material comprising at least one adsorbing/adsorptive polymer, or at least one non-adsorbing/non-adsorptive polymer, or at least one adsorbing/adsorptive polymer together with at least one non-adsorbing/non-adsorptive polymer, wherein said at least one first adsorbent and said at least one second adsorbent are subsequently used, either provided in chromatographic columns or in means configured to perform in batch separation, or provided within a combination of chromatographic columns, or provided in means configured to perform in batch separation, and/or filtration.

22. The method according to claim 20, wherein the target compound is characterized by a hydrodynamic radius Rₕᵢ and the at least one impurity compound is characterized by a hydrodynamic radius R_{h2,} wherein Rₕᵢ>Rₕ₂, the method comprising the following steps (i) and (iii) to (iv) and optionally step (v):
(i) providing the at least one composite material, the at least one composite material or at least one polymeric layer of the polymeric mesh being characterized by a pore size exclusion limit Rₕᵢ such that Rₕ₂<Rₕᵢ and Rill>Rhi;
(iii) contacting the at least one composite material with the feedstock for a time sufficient to allow retaining the at least one impurity compound in the at least one composite material and excluding the target compound from the at least one composite material;
(iv) separating the at least one composite material containing the retained at least one impurity compound from the feedstock containing the excluded target compound in order to obtain a purified feedstock;
(v) optionally, isolating the target compound from the purified feedstock.

23. A method for setting the exclusion limit Rₕᵢ of a composite material or at least one polymeric layer of a polymeric mesh as defined in claim 1, wherein the target compound is characterized by a hydrodynamic radius Rₕᵢ and the at least one impurity compound is characterized by a hydrodynamic radius Rh2, wherein Rhi>Rh2, the method comprising the following steps (i) to (ii):
(i) providing said at least one composite material, the composite material or at least one polymeric layer of the polymeric mesh respectively exhibit a pore size exclusion limit Rₕᵢ which can be set variably;
(ii) adapting the variable pore size exclusion limit Rₕᵢ to the hydrodynamic radii Rₕᵢ and Rₕ₂ such that Rₕ₂<Rₕᵢ and Rₕᵢ>Rₕ.

24. Method of claim 23, wherein said setting in step (i) or said adapting in step (ii) or said setting in step (i) and said adapting in step (ii) is performed by one or more of the following: varying the structure of the at least one polymeric layer of the polymeric mesh, selecting the crosslinker used to generate a crosslinked at least one polymeric layer, selecting the degree of cross-linkage of the at least one polymeric layer, controlling the degree of swelling of the at least one polymeric layer by varying the solvent for the preparation and the use of the at least one polymeric layer, particularly varying the pH of the solvent and thus the degree of protonation of the at least one polymeric layer, and controlling the concentration and the immobilized amount of the at least one polymeric layer of the polymeric mesh within said at least one composite material.

25. The composite material according to claim 1, wherein the target compound comprises a biopolymer or a microorganism, the biopolymer being selected from the group consisting of peptides, proteins, glycoproteins, lipoproteins, and a combination of two or more thereof, and the microorganism being selected from viruses, bacteria, cells and fragments thereof; and a combination of two or more thereof, and wherein the impurity compound comprises host cell proteins (HCP), DNA, RNA or other nucleic acid, or a combination of two or more thereof, and optionally further comprising albumins, endotoxins detergents and microorganisms, or fragments thereof, or a combination of two or more thereof.

26. Use of a composite material as defined in claim 1 for recovering a target compound from a solution or suspension, either being a feedstock, or being the eluate of a preceding purification step, said solution or suspension comprises said target compound, wherein the target compound comprises a biopolymer or a microorganism, the biopolymer being selected from the group consisting of peptides, proteins, glycoproteins, lipoproteins, and the microorganism being selected from viruses, bacteria, cells and fragments thereof; or a combination of two or more thereof, and further comprises at least one impurity compound, and optionally further comprising albumins, endotoxins detergents and microorganisms, or fragments thereof, or a combination of two or more thereof.

27. A method for the selective coating of a porous support material, the method comprising:
filling the support material with a solvent which is water-immiscible, whereas the external surface of the support material becomes coated with a functional polymer in aqueous solution, or
filling the support material with an aqueous solution whereas the external surface of the support material becomes coated with a functional polymer in a solvent which is water-immiscible.

28. (canceled)

29. A method for the selective derivatisation of at least one functional group of a polymer comprised by a polymeric mesh, the method comprising:
filling the polymeric mesh with a solution of a derivatisation reagent in a solvent which is water-immiscible, wherein the volume outside of the mesh comprises an aqueous solvent, or
filling the polymeric mesh with a solution of a derivatisation reagent in a solvent, wherein the volume outside of the mesh is not containing a liquid, or
filling the polymeric mesh with an aqueous solvent, wherein the volume outside of this mesh, but in contact with its boundary surface, comprises a solution of a derivatisation reagent in a solvent which is water-immiscible.

30-31. (canceled)
